(19) United States
(12) Patent Application Publication
(10) Pub. No.: US 2011/0166224 A1
(43) Pub. Date: Jul. 7, 2011

(54) DIACYLGLYCEROL RICH FATS, OILS AND FUNCTIONAL FOODS

(76) Inventors: Ganesh M. Kishore, Saint Louis, MO (US); Riccardo G. Locascio, San Francisco, CA (US)

(21) Appl. No.: 12/981,302

(22) Filed: Dec. 29, 2010

Related U.S. Application Data
(63) Continuation-in-part of application No. PCT/US2009/053442, filed on Aug. 11, 2009.
(60) Provisional application No. 61/087,926, filed on Aug. 11, 2008.
(60) Provisional application No. 61/087,991, filed on Aug. 11, 2008.

(51) Int. Cl. A61K 31/23; A23D 9/00; A21D 13/00; A21D 10/00; A21D 13/08; A23C 9/123; A23L 1/187; A23C 19/00; A23D 7/00; A23C 15/12; A23L 1/24; A61P 9/12; A61P 9/00; A61P 3/10; A61P 3/00; A61P 3/06; A61P 9/10

(52) U.S. Cl. 514/547; 426/601; 426/607; 426/560; 426/549; 426/556; 426/572; 426/583; 426/579; 426/582; 426/589; 426/603; 426/581; 426/605; 426/2; 426/442; 426/417

(57) ABSTRACT

Disclosed is a fat or oil useful for cooking applications which includes from 10 to 90% by weight diacylglycerols, and has a SFI of at least 15% at room temperature. In particular embodiments, the fat or oil is derived from palm oil, palm kernel oil, coconut oil, sunflower oil, soybean oil, corn oil, rapeseed oil, grape seed oil, rice bran oil, sesame oil, and peanut oil, or any combination thereof. In some cases, the fat or oil exhibits health benefits including lowered serum LDL, raised serum HDL, lowered total serum cholesterol, reduced risk of metabolic syndrome, reduced risk of diabetes mellitus, enhanced fetal health, enhanced insulin sensitivity, reduced risk of hypertension, and enhanced resistance to obesity per unit of consumption. Food compositions and methods of health enhancement utilizing the fats and oils of the invention are also disclosed.

### CROSS-REFERENCE TO RELATED APPLICATIONS

The present application claims priority to U.S. Prov. App. Ser. No. 61/087,926 (filed Aug. 11, 2008); U.S. Prov. App. Ser. No. 61/087,991 (filed Aug. 11, 2008); and PCT/US2009/053442 (filed Aug. 11, 2009), each of which are incorporated herein by reference in their entirety.

### TECHNICAL FIELD

The present teachings relate to compositions and methods for making and using health-promoting diacylglycerol-rich semi-solid fats and oils as functional foods. The semi-solid fats and oils can be derived from any source, and the diacylglycerol molecules can be obtained using any known methods. In some cases, the diacylglycerol-rich fats and oils can be derived from palm and other tropical vegetable oils, and may be optionally combined with sunflower, soy, corn, rapeseed and other temperate vegetable oils of high palmitic and/or high stearic acid content. The fats and oils can be used for cooking, as well as in food preparations, medicinal supplements, pharmaceuticals, cosmetics and other relevant applications. The semi-solid fats and oils can comprise both 1,2 and 1,3 diacylglycerol molecules, and can comprise fatty acids of chain lengths comprising between 8-22 carbons. The fatty acids can comprise saturated or partially saturated fatty acids.

### BACKGROUND

There is a demand in many food sectors to find healthier saturated fats to replace trans fats. Trans fats are industrially created by partially hydrogenating plant oils to create more saturated, higher melting point solid fats. Trans fatty acids (“TFAs”) are produced when oils and fats containing unsaturated fatty acids are “hydrogenated” in the presence of a catalyst. TFAs are the geometrical isomers of unsaturated fatty acids containing at least one double bond in the trans configuration. This trans configuration of the double bond imparts physical properties including reduced fluidity of the fat, thereby increasing its melting point. Hydrogenation primarily increases the melting range of unsaturated fats and thereby enables their incorporation into many solid fat formulations. When an unsaturated fat or oil is fully hydrogenated, all the unsaturated fatty acids are converted into their saturated analogues. Since unsaturation in most vegetable oils is largely 18-carbon fatty acids, namely oleic (18:1, n-9), linoleic (18:2, n-6) and linolenic (18:3, n-3), full hydrogenation of such oils would result in a high melting block of fat containing stearic acid (18:0). Partial hydrogenation, in the presence of catalysts, results in the formation of TFAs. Thus, partial hydrogenation of liquid oils has been a tool of choice to enable their use in solid fat formulations. These trans fats are used for applications such as deep frying and baking, while extending the shelf life of products of these processes. TFAs are widely distributed in foods containing traditional margarine, bakery and frying fats, vegetable shortenings, and vanaspati.

Since their introduction into the human diet and until the early 1990s, partially hydrogenated fats containing TFA were advocated as the preferred fatty acid base for solid fats, especially margarines. They were initially designed to replace butterfat, and with advancements in our knowledge about the adverse impacts of saturated fatty acids (“SFA”) on cardiovascular disease (“CVD”) risk, TFAs were prominently touted as a safe alternative. However, health authorities worldwide, and in particular the U.S. Food & Drug Administration (FDA), have recently recommended that consumption of trans fats be reduced to zero or to trace amounts due to their ability to increase coronary heart disease by raising levels of “bad” low-density lipoprotein (“LDL”) cholesterol and lowering “good” high-density lipoprotein (“HDL”) cholesterol. A study by Mensink and Katan suggested that TFA increased total and LDL cholesterol and decreased the beneficial HDL cholesterol following the consumption of a high-TFA diet (Mensink R P, Katan M B. N. Engl. J. Med. 1990 323:439-445). Repeatedly, studies have established that TFA containing diets could be worse than the SFA-rich diets they were designed to replace. A Nurses Health Study elucidated the effects of a TFA containing diet using epidemiological data from 85,095 women, establishing an association between TFA and the incidence of non-fatal myocardial infarction from coronary heart disease. A positive and significant association between TFA and CVD was apparent. Foods that were major sources of TFA, including margarine and cookies, also revealed a positive correlation. Relative risk for CVD was increased by 27% as a result of TFA consumption.

Other studies showed adverse effects of TFAs on serum markers of inflammation, including related enzymatic activity, and immune function. See Baer D J, Judd J T, Clevidence B A, et al. Dietary fatty acids affect plasma markers of inflammation in healthy men fed controlled diets: a randomized crossover study, Am J Clin Nutr 2004; 79:969-73; de Roos N M, Schouten E G, Scheek L M, et al. Replacement of dietary saturated fat with trans fat reduces serum paraoxonase activity in healthy men and women, Metabolism 2002; 12:1534-7; and Han S N, Leka L S, Lichtenstein A H, et al. Effect of hydrogenated and saturated, relative to polyunsaturated, fat on immune and inflammatory responses of adults with moderate hypercholesterolemia, J Lipid Res 2002; 43:445-52. These studies established a clear association of TFA consumption with increased incidence and death from CVD. It was estimated that almost 80,000 deaths in the U.S. alone were associated with continued consumption of foods rich in TFA. Other recent studies have implicated TFA-rich diets with increased risk and incidence of diabetes. Other concerns include adverse effects of TFA on cardiac arrhythmia and in pregnant women, underlying implications for the health of the developing fetus since TFA competes with essential fatty acids during fetal development.

### SUMMARY

Saturated fats are solid at room temperature and provide foods with taste and structural functionalities. Excessive consumption of saturated and semi-saturated fats has been determined to raise LDL cholesterol, and has been correlated with hyperlipidemia, hypercholesteremia, hyperglycemia, insulin resistance, postprandial lipemia and other aspects of metabolic syndrome. Food formulators are unable to further reduce the amount of saturated fats per serving without sacrificing many of the structural and taste characteristics typical of solid fats. Trans-fats, which are also solid or semi-solid at room temperature and possess structural and functional benefits similar to saturated fats, have been widely used in the food industry. Epidemiological data has shown, however, that trans-fats increase the risk of CVD. Currently, all solid and semi-solid fats, which comprise of saturated and trans-fats, are indicated as negative for human health. The food industry lacks saturated and trans-fats that are simultaneously neutral or beneficial to human health and that can provide foods with important taste, structural and functional attributes. Provided herein are saturated fat compositions in the form of diacylglycerols (e.g., palm diacylglycerols or palm kernel diacylglycerols) with the taste, structural and functional attributes of a solid, saturated fat but with the unexpected health benefits of reducing triglycerides (TG), total cholesterol (TC), and LDL-C cholesterol.

The present teachings include diacyglycerol (“DAG”)-based semisolid fat and oil compositions.

In accordance with an embodiment of this aspect, the DAG-based semi-solid fat and oil compositions can be derived from a plant selected from the group consisting of palm, palm kernel, coconut, other tropical plants, temperate plants and algae.

In accordance with a further embodiment, the DAG-based semisolid fat and oil compositions can be derived from non-plant sources. In a further aspect of the embodiment, the non-plant source can be a fish.

The present teachings include methods for cooking and food preparation using the DAG-based semi-solid fat and oil compositions of the present disclosure.

In accordance with a further aspect, foods comprising the DAG-based semi-solid fat and oil compositions are provided.

In accordance with yet another aspect, cooking fats and oils comprising the DAG-based semi-solid fat and oil compositions are provided.

In accordance with yet another aspect, methods for managing metabolic syndrome and cardiovascular disorders and/or improving postprandial and fasting blood lipid levels are provided.

In accordance with an embodiment of the present disclosure, a semi-solid fat or oil comprising DAG derived from a tropical oil is provided. In a further aspect of this embodiment, the oil is selected from the group consisting of palm oil, palm kernel oil, coconut oil and other oils, including but not limited to, oils with a high stearic acid content.

In a further embodiment, the fat or oil exhibits beneficial health effects when ingested by a mammal. In an aspect of this embodiment, the beneficial health effects comprise amelioration of a disease state. In a further aspect of this embodiment, the disease state is selected from the group consisting of hyperlipemia, hypercholesteremia, hyperglycemia, insulin resistance, postprandial lipemia, and other aspects of metabolic syndrome.

In a further aspect of this embodiment, the beneficial health effects are selected from the group consisting of lowered serum LDL, raised serum HDL, lowered total serum cholesterol, reduced risk of metabolic syndrome, reduced risk of diabetes, enhanced fetal health, enhanced insulin sensitivity, reduced risk of hypertension, reduction of inflammatory biomarkers related to obesity and enhanced resistance to obesity.

In some aspects, an inflammatory biomarker related to obesity can be selected from the group consisting of cytokines, C-reactive protein (CRP), interleukin-6 (IL-6), monocyte chemoattractant protein-1 (MCP-1), tumor necrosis factor alpha (TNF-α), interleukin-18 (IL-18), interleukin-10 (IL-10), serum amyloid A (SAA), fibrinogen, intercellular adhesion molecule-1 (ICAM-1), lipoprotein-associated phospholipase-A2 (Lp-PLA2), myeloperoxidase, CD40 ligand, osteoprotegerin, P-selectin, and tumor necrosis factor receptor-II.

In a further aspect of this embodiment, the beneficial health effects are selected from the group consisting of a reduction in weight of the mammal and a reduction in intracellular inflammatory markers.

In a further embodiment, a fat or oil as described above may be provided that additionally comprises medium-chain diglycerides.

In an embodiment of the present disclosure, a fat or oil useful for cooking applications comprising from 10 to 90% DAG, comprising at least 15% solids at room temperature is provided.

In an aspect of this embodiment, the fat or oil comprises from 20 to 70% DAG. In a further aspect of this embodiment, the fat or oil comprises from 25 to 60% DAG. In a further aspect of this embodiment, the fat or oil comprises from 30 to 50% DAG.

In a further aspect of this embodiment, the fat or oil comprises from 20% to 60% solids at room temperature. In a further aspect of this embodiment, the fat or oil comprises from 22% to 50% solids at room temperature.

In a further aspect of this embodiment, the DAG content is derived from a plant selected from the group consisting of palm, palm kernel, coconut, other tropical plants, non-tropical plants, vegetables and algae. In a further aspect of this embodiment, the DAG content is derived from an oil selected from the group consisting of palm, palm kernel, coconut and high-stearate vegetable oil, or any combination thereof. In a further aspect of this embodiment, the DAG content is derived from palm oil. In accordance with a further embodiment, the DAG-based semisolid fat and oil compositions can be derived from non-plant sources.

In a further aspect of this embodiment, the saturated fat content of the DAG component has been increased from 5% to 30% over the parent stock from which the DAG component is derived.

In a further aspect of this embodiment, the saturated fat content of the DAG component has been increased from 15% to 30% over the parent stock from which the DAG component is derived.

In a further aspect of this embodiment, the DAG component of the fat or oil comprises at least 25% 1,3-DAG.

In a further aspect of this embodiment, dietary consumption of the fat or oil, or foods cooked or prepared using said fat or oil, provides one or more of the health benefits selected from the group consisting of lowered serum LDL, raised serum HDL, lowered total serum cholesterol, reduced risk of metabolic syndrome, reduced risk of diabetes, enhanced fetal health, enhanced insulin sensitivity, reduced risk of hypertension, reduction of inflammatory biomarkers related to obesity, and enhanced resistance to obesity per unit of consumption.

In a further aspect of this embodiment, the fat or oil further comprises one or more of the additional ingredients selected from the group consisting of phytosterol, and phytostanol.

In a further aspect of this embodiment, the composition further comprises phytosterol.

In a further embodiment, a food composition is provided that comprises a fat or oil component of any of the above embodiments, wherein the food composition is formulated to comprise one of the foodstuffs selected from the group consisting of shortening, bakery fat, frying fat, cocoa-butter equivalent, cocoa-butter replacer, margarine, and vanaspati. In a further aspect of this embodiment, the food composition is formulated to comprise shortening.

In a further embodiment, a food composition is provided that comprises a prepared food cooked or prepared using the food composition of any of the above embodiments, wherein the food composition is selected from the group consisting of cakes, breads, sweet dough, cream filling, ice cream, granola bars, pastry, non-dairy fats, coating fats, deep fat fries, shortening, coca-butter substitutes, specialty fats and bakery fats.

In a further aspect of this embodiment, the food composition exhibits one or more of the enhanced characteristics selected from the group consisting of enhanced shelf-stability, enhanced emulsion stability, reduced brittleness, enhanced spreadability, enhanced melt-in-the-mouth sensation, higher melting-point, reduced trans-fatty acid content per unit of solids consumed, reduced PUFA content per unit of solids consumed, reduced susceptibility to oxidation, enhanced texture, enhanced palatability, enhanced lubricity, and enhanced air trapping capacity. In particular, the food composition exhibits one or more of the enhanced characteristics selected from the group consisting of increased palatability, mouth feelings and sensory attributes of non-fat or reduced fat products. In a further aspect of this embodiment, the food composition exhibits enhanced shelf-stability.

In an embodiment of the present disclosure, a method is provided for providing one or more of the health benefits selected from the group consisting of lowered serum LDL, raised serum HDL, lowered total serum cholesterol, reduced risk of metabolic syndrome, reduced risk of diabetes, enhanced fetal health, enhanced insulin sensitivity, reduced risk of hypertension, reduction of inflammatory biomarkers related to obesity and enhanced resistance to obesity per unit of consumption to a subject comprising administering to said subject a food composition in accordance with any of the embodiments above.

In a further aspect of this embodiment, the health benefit provided comprises reduction of inflammatory biomarkers related to obesity.

In a further aspect, the oils having a high stearic acid content comprise 12% or more stearic acid by weight.

In a further aspect, the oils having a high stearic acid content are selected from the group consisting of sunflower oil, soybean oil, corn oil, rapeseed oil, grape seed oil, rice bran oil, sesame oil, shea butter, cocoa butter and peanut oil.

In a further aspect of any of these embodiments, the DAG component of the fat or oil comprises 40%-99% 1,3-DAG.

In a further aspect of any of these embodiments, the DAG component of the fat or oil comprises 50%-95% 1,3-DAG.

In a further aspect of any of these embodiments, the DAG component of the fat or oil comprises 60%-90% 1,3-DAG.

In a further aspect of any of these embodiments, the DAG component of the fat or oil comprises at least 70% 1,3-DAG.

In a further aspect of any of these embodiments, the DAG component of the fat or oil comprises 40%-99% 1,2-DAG.

In a further aspect of any of these embodiments, the DAG component of the fat or oil comprises 50%-95% 1,2-DAG.

In a further aspect of any of these embodiments, the DAG component of the fat or oil comprises 60%-90% 1,2-DAG.

In a further aspect of any of these embodiments, the DAG component of the fat or oil comprises at least 70% 1,2-DAG.

In a further embodiment, the DAG comprises SFAs of 8-22 carbons.

In a further aspect of this embodiment, the DAG comprises SFAs of 8-18 carbons.

In a further aspect of the embodiment, the SFAs can be derived from any source.

In a further aspect of this embodiment, the SFAs can be derived from plants selected from the group consisting of soy, sunflower, canola/OSR (oilseed rape), shea butter and cocoa butter.

In a further aspect of this embodiment, the SFAs can be derived from a source that has been modified to contain high SFA levels.

In an additional embodiment, the DAG comprises at least one unsaturated fatty acid at the 1, 2, or 3 position.

In a further aspect of this embodiment, the at least one unsaturated fatty acid is selected from the group consisting of an 18:1, 18:3, 18:4, 20:3, 20:4, 20:5, and 22:6 fatty acid.

In a further aspect of the embodiment, the at least one unsaturated fatty acid is selected from the group consisting of an omega 3 and an omega 6 fatty acid.

In a further aspect of any of these embodiments, the unsaturated fatty acids may be derived from any source. Fish, alga and plants are provided as non-limiting examples of a source of unsaturated fatty acids.

In a further aspect of any of these embodiments, the SFA component of the fat or oil comprises 15%-99% SFA.

In a further aspect of any of these embodiments, the SFA component of the fat or oil comprises 50%-99% SFA.

In a further aspect of any of these embodiments, the SFA component of the fat or oil comprises 60%-99% SFA.

In a further aspect of any of these embodiments, the SFA component of the fat or oil comprises 70%-99% SFA.

In a further aspect of any of these embodiments, the SFA component of the fat or oil comprises 80%-99% SFA.

In a further aspect of any of these embodiments, the SFA component of the fat or oil comprises 60%-99% SFA.

In a further aspect of any of these embodiments, the SFA component of the fat or oil comprises 15% to 50% SFA.

In a further aspect of any of these embodiments, the SFA component of the fat or oil comprises 20% to 50% SFA.

In a further aspect of any of these embodiments, the SFA component of the fat or oil comprises 30% to 50% SFA.

In a further aspect of any of these embodiments, the SFA component of the fat or oil comprises 40% to 50% SFA.

In a further aspect of any of these embodiments, the SFA component of the fat or oil comprises at least 15% SFA.

In a further aspect of these embodiments, the percentage of SFAs is the number of fatty acids which are SFAs divided by the total number of fatty acids, times 100.

In further aspects of the embodiments, there is provided a food composition which exhibits one or more of the enhanced characteristics selected from the group consisting of increased palatability, mouth feel, and sensory attributes of non-fat or reduced fat products.

In further aspects of the embodiments, a semi-solid fat or oil comprising 10 to 90% DAG blended with monounsaturated fatty acids (MUFAs), polyunsaturated fatty acids (PUFAs), medium-chain fatty acids and a combination of one or more thereof is provided.

In a further aspect of the embodiment, the oils and fats blended with the DAG-containing compositions are derived from a source selected from the group consisting of fish, algae, vegetables and any combination thereof.

In an additional aspect of the embodiment, the oils and fats blended with the DAG-containing compositions are derived from a source selected from the group consisting of palm, coconut, any tropical oils, sunflower, corn, soybean, rapeseed and canola oils.

In another aspect of the embodiment, the oils and fats blended with the DAG-containing compositions comprise one or more of 18:1, 18:2, 18:3 (both omega 3 and omega 6), 18:4, 20:3, 20:4, 20:5 and 22:6 omega 3 fatty acids.

In one embodiment, the oil or fat blended with the DAG-containing compositions comprises gamma-linolenic acid.

In an additional embodiment, the oil or fat blended with the DAG-containing compositions comprises stearidonic acid.

In another aspect, this disclosure features a fat composition that includes from about 20% to about 100% by weight of diacylglycerols derived from a tropical oil. The tropical oil can be selected from the group consisting of palm oil, palm kernel oil, cocoa oil, shea oil, and coconut oil. In one embodiment, the tropical oil is selected from palm oil and palm kernel oil. The palm oil can be a fraction of palm oil selected from palm olein, palm stearine, fractionated palm olein, palm mid-fraction, and mixtures thereof. The palm kernel oil can be a fraction of palm kernel oil selected from palm kernel olein, palm kernel stearin, and mixtures thereof.

This document also features an oil composition that includes from about 20% to about 100% by weight of diacylglycerols derived from a temperate oil, the temperate oil having a saturated fatty acid content of at least 15%. The temperate oil can be selected from the group consisting of corn oil, soybean oil, canola oil, and sunflower oil.

Compositions described herein can exhibit beneficial health effects (e.g., lowered serum LDL and/or lowered total serum cholesterol) when an effective amount of the composition is ingested by a mammal for at least 4 weeks. The composition can have a solid fat index (SFI) of at least 15% (e.g., from about 20% to about 60% or from about 22% to about 50%) at room temperature. The composition can include from about 40% to about 100% (e.g., about 70% to about 100%, or about 85% to about 100%) by weight of the diacylglycerols. The diacylglycerols can include at least 25% (e.g., at least 50%, 60%, 70%, 80%, 90%, 95%, 99%, or 100%) by weight 1,3-diacylglycerols. The diacylglycerols can include from about 50 to about 80% by weight 1,3-diacylglycerols. The composition can include less than about 1% by weight of one or more of a phytosterol, phytostanol, phytosterol ester, or phytostanol ester. The composition can include no detectable level of one or more of a phytosterol, phytostanol, phytosterol ester, or phytostanol ester.

This disclosure also features a food composition that includes any of the compositions described herein. The food composition can be selected from the group consisting of: cakes, pies, breads, muffins, crackers, sweet dough, pastry, cream filling, yogurt, pudding, cream cheese, spreads, salad dressing, margarines, butter, mayonnaise, frozen foods, ice cream, frozen desserts, frozen yogurt, smoothies, peanut butter, granola, bars, cookies, and medical foods. The medical food can be a meal replacement bar, meal replacement beverage, semi-solid beverage for enteric nutrition, or a semi-solid beverage for tube feeding. The food composition can exhibit one or more enhanced characteristics selected from the group consisting of enhanced shelf-stability, enhanced emulsion stability, reduced brittleness, enhanced spreadability, enhanced melt-in-the-mouth sensation, higher melting-point, reduced trans fatty acid content per unit of solids consumed, reduced susceptibility to oxidation, enhanced texture, enhanced palatability, enhanced lubricity, and enhanced air trapping capacity. For example, the food composition can exhibit enhanced shelf stability.

In another aspect, this document features a fatty acid composition that includes from about 20 to about 95% by weight of diacylglycerols, wherein the diacylglycerols include 1,2-diacylglycerols and 1,3-diacylglycerols, and wherein from about 15% to about 40% by weight of the diacylglycerols include at the 2 position, an unsaturated fatty acid, and wherein from about 25% to about 100% by weight of the diacylglycerols include at the 1(3) position, a saturated fatty acid having a chain length of 6-18 carbon atoms. The unsaturated fatty acid can be from a source selected from the group consisting of fish oil, algae, a temperate oil, and combinations thereof. The unsaturated fatty acid can be selected from the group consisting of 18:1, 18:2, 18:3, 18:4, 20:3, 20:4, 20:5 and 22:6 fatty acids. The unsaturated fatty acid can include gamma-linolenic acid or stearidonic acid. The saturated fatty acid can be from a source selected from the group consisting of palm, palm kernel, coconut, sunflower, corn, soybean, canola, and high stearate oils.

This document also features a fat composition that consists essentially of from about 60 to about 100% by weight of diacylglycerols, wherein the fatty acid content of the diacylglycerols consists essentially of from about 40 to about 50% by weight 16:0 fatty acid, from about 3 to about 6% by weight 18:0 fatty acid, from about 30 to about 45% by weight 18:1 fatty acid, and from about 7 to about 12% by weight 18:2 fatty acid.

In another aspect, this document features a fat composition that consists essentially of from about 60 to about 100% by weight of diacylglycerols, wherein the fatty acid content of the diacylglycerols consists essentially of from about 45 to about 55% by weight 12:0 fatty acid; from about 15 to about 20% by weight 14:0 fatty acid; from about 6 to about 12% by weight 16:0 fatty acid; and from about 10 to about 20% by weight 18:1 fatty acid.

This document also features a fat composition that consists essentially of from about 60 to about 100% by weight of diacylglycerols derived from palm oil, wherein the fatty acid content of the diacylglycerols consists essentially of from about 40 to about 50% by weight 16:0 fatty acid, from about 3 to about 6% by weight 18:0 fatty acid, from about 30 to about 45% by weight 18:1 fatty acid, and from about 7 to about 12% by weight 18:2 fatty acid. The composition can consist essentially of from about 85 to about 90% by weight of the diacylglycerols. The composition can have a SFI at least 15% at room temperature. The diacylglycerols can include from about 60 to about 80% by weight 1,3-diacylglycerols and from about 20 to about 40% by weight 1,2-diacylglycerols. The composition can consist essentially of less than about 10% by weight monoacylglycerols. The composition can consist essentially of less than about 15% by weight triacylglycerols. The fatty acid content of the diacylglycerols can include about 46% by weight 16:0 fatty acid, about 4% by weight 18:0 fatty acid, about 36% by weight 18:1 fatty acid, and about 9% by weight 18:2 fatty acid.

This document also features a fat composition that consists essentially of from about 60 to about 100% by weight of diacylglycerols derived from palm kernel oil, wherein the fatty acid content of the diacylglycerols comprises from about 45 to about 55% by weight 12:0 fatty acid; from about 15 to about 20% by weight 14:0 fatty acid; from about 6 to about 12% by weight 16:0 fatty acid; and from about 10 to about 20% by weight 18:1 fatty acid. The composition can consist essentially of from about 75 to about 85% by weight of the diacylglycerol. The composition can have a SFI of at least 15% at room temperature. The diacylglycerols can include from about 60 to about 80% by weight 1,3-diacylglycerols and from about 20 to about 40% by weight 1,2-diacylglycerols. The composition can consist essentially of less than about 10% by weight monoacylglycerols. The composition can consist essentially of less than about 25% by weight triacylglycerols. The diacylglycerols can include about 47% by weight 12:0 fatty acid; about 18% by weight 14:0 fatty acid; from about 9% by weight 16:0 fatty acid; from about 16% by weight 18:1 fatty acid.

In another aspect, this document features a method of lowering total serum cholesterol or serum LDL in a mammal. The method includes administering to the mammal an amount of a composition described herein to provide at least 15 g/day of diacylglycerols to the mammal and for a period of time sufficient to lower the total serum cholesterol or serum LDL. The diacylglycerols can be derived from a tropical oil such as palm oil or palm kernel oil. The palm oil can be a fraction of palm oil selected from palm olein, palm stearine, fractionated palm olein, palm mid-fraction, and mixtures thereof. The palm kernel oil can be a fraction of palm kernel oil selected from palm kernel olein, palm kernel stearin, and mixtures thereof. The composition can be administered as an ingredient in a foodstuff. The composition can be administered as an ingredient in a medical food selected from the group consisting of a meal replacement bar, meal replacement beverage, semi-solid beverage for enteric nutrition, and a semi-solid beverage for tube feeding. The foodstuff can be selected from the group consisting of: cakes, pies, breads, muffins, crackers, sweet dough, pastry, cream filling, yogurt, pudding, cream cheese, spreads, salad dressing, margarines, butter, mayonnaise, frozen foods, ice cream, frozen desserts, frozen yogurt, smoothies, peanut butter, granola, bars, and cookies. The ingredient can be selected from the group consisting of: a shortening, bakery fat, frying fat, coating fat, non-dairy fat, cocoa-butter equivalent, butter, margarine, and vanaspati ghee. The ingredient can include less than about 1% (e.g., a non-detectable level) by weight phytosterol, phytostanol, phytosterol ester, or phytostanol ester.

The mammal can be diagnosed as having a cardiovascular disorder (e.g., a cardiovascular disorder selected from one or more of: hypertriglyceridemia, hypercholesterolemia, other hyperlipidemias, hyperglycemia, hyperinsulinemia, arteriosclerosis, atherosclerosis, arteriolosclerosis, angina pectoris, thrombosis, myocardial infarction, and hypertension). The mammal can be diagnosed as having type II diabetes or as having a metabolic syndrome. The mammal can have a serum level of LDL ranging from about 120 to about 175 mg/dL prior to administration of the composition. The lowering can occur after administering the composition for at least twelve weeks. The method can include administering to the mammal an amount of the composition to provide at least 30 g/day (e.g., at least 40 g/day) of diacylglycerols.

This document also features a method for producing a food product. The method includes substituting diacylglycerols derived from palm oil, a palm kernel oil, or a mixture thereof for about 5% to about 100% of a hydrogenated fat or oil in the food product.

In another aspect, this document features a food product that includes a fat composition. The fat composition includes from about 20% to about 100% by weight of diacylglycerols derived from a tropical oil, wherein the prepared food product lowers one or more of total serum cholesterol and serum LDL in a mammal. The fat component of the food product can consist essentially of the fat composition. The food product can be selected from the group consisting of baked prepared foods, dairy products, and blended food products. The baked prepared foods, dairy products, and blended food products can be selected from the group consisting of cakes, pies, breads, muffins, crackers, sweet dough, pastry, cream filling, yogurt, pudding, cream cheese, spreads, salad dressing, margarines, butter, mayonnaise, frozen foods, ice cream, frozen desserts, frozen yogurt, smoothies, peanut butter, granola, bars, and cookies.

In another aspect, this document features a method of making a composition. The method includes reacting free fatty acids and glycerol with one or more sn-1,3-regiospecific lipases, wherein the free fatty acids are from a tropical oil or a temperate oil having a saturated fatty acid content of at least 15%; and purifying the resulting diacylglycerol products to obtain the composition having at least 20% diacylglycerols. The one or more sn-1,3-regiospecific lipases can be immobilized. The tropical oil can be selected from palm oil and palm kernel oil. The temperate oil can be selected from corn, soybean, canola and sunflower oil.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

These and other features, aspects and advantages of the present teachings will become better understood with reference to the following description, examples and appended claims.

### DESCRIPTION OF THE DRAWINGS

Those of skill in the art will understand that the drawings, described below, are for illustrative purposes only. The drawings are not intended to limit the scope of the present teachings in any way.

FIG. 1 is a schematic of the metabolic pathway of triacylglycerol (TAG) (1A) vs. DAG (1B).

FIG. 2 is a diagram of the chemical structure of a) a TAG, b) a representative 1,2 diacylglycerol, and c) a representative 1,3 diacylglycerol.

FIG. 3 is a graph showing the solid fat index of several compositions at various temperatures.

FIG. 4 is a pictorial representation of a protocol testing beneficial health effects of DAG-containing compounds.

FIG. 5 is a bar graph of the mean (±SEM) total cholesterol (TC) levels at baseline and after consumption of the control or DAG diets (*Significant change from baseline, p<0.05 (ANOVA)).

FIG. 6 is a bar graph of the mean (±SEM) low density lipoprotein-cholesterol (LDL-C) levels at baseline and after consumption of the control or DAG diets (*Significant change from baseline, p<0.05 (ANOVA)).

FIG. 7 is a bar graph of the mean (±SEM) high density lipoprotein-cholesterol (HDL-C) levels at baseline and after consumption of the control or DAG diets (*Significant change from baseline, p<0.05 (ANOVA)).

FIG. 8 is a line graph showing TC (mg/dL) response after palm DAG oil or control (palm oil) consumption.

FIG. 9 is a line graph showing LDL-C (mg/dL) response after palm DAG oil or palm oil consumption.

FIG. 10 is a line graph showing HDL-C (mg/dL) response after palm DAG oil or palm oil consumption.

FIG. 11 is a line graph showing the mean (±SEM) TG response to palm DAG oil and control (palm oil), over the time course of 6 hours.

FIG. 12 is a line graph showing the mean (±SEM) adjusted TG response to palm DAG oil and control (palm oil), over the time course of 6 hours.

FIG. 13A is a line graph showing the TC response to palm DAG oil and control (palm oil), over the time course of 8 hours; 13B is a bar graph illustrating the TC AUC for both groups.

FIG. 14A is a line graph showing the HDL-C response to palm DAG oil and control (palm oil), over the time course of 8 hours; 14B is a bar graph illustrating the HDL-C AUC for both groups.

FIG. 15A is a line graph showing the LDL-C response to palm DAG oil and control (palm oil), over the time course of 8 hours; 15B is a bar graph illustrating the LDL-C AUC for both groups.

FIG. 16A is a line graph showing the TG response to palm DAG oil and control (palm oil), over the time course of 8 hours; 16B is a bar graph illustrating the TG AUC for both groups.

FIG. 17 is a schematic of a method for producing DAGs.

### DETAILED DESCRIPTION

Saturated fats are solid at room temperature and provide foods with taste and structural functionalities. Excessive consumption of saturated and semi-saturated fats has been determined to raise LDL cholesterol, and has been correlated with hyperlipidemia, hypercholesteremia, hyperglycemia, insulin resistance, postprandial lipemia and other aspects of metabolic syndrome. Food formulators are unable to further reduce the amount of saturated fats per serving without sacrificing many of the structural and taste characteristics typical of solid fats. Trans-fats, which are also solid or semi-solid at room temperature and possess structural and functional benefits similar to saturated fats, have been widely used in the food industry. Epidemiological data has shown, however, that trans-fats increase the risk of CVD. Currently, all solid and semi-solid fats, which comprise of saturated and trans-fats, are indicated as negative for human health. The food industry lacks saturated and trans-fats that are simultaneously neutral or beneficial to human health and are able to provide foods with important taste, structural and functional attributes. Provided herein are saturated fat compositions in the form of diacylglycerols (e.g., palm diacylglycerols or palm kernel diacylglycerols) with the taste, structural and functional attributes of a solid, saturated fat but with the unexpected health benefits of reducing triglycerides (TG), total cholesterol (TC), and LDL-C cholesterol.

Applicant's DAG-rich oils and fats provide enhanced nutritional value because of their differential metabolism in the body. Additionally, their reduction of saturated fats provides superior health attributes versus saturated fat-containing oil, e.g., reducing postprandial LDL and triglyceride (“TAG”) levels, yet retains the important physical properties of solid fats needed to replace trans fats in foods and cooking fats and oils. These DAG-rich oils retain excellent physical properties for superior domestic and commercial cooking and frying oil, as well as for incorporation into other foodstuffs.

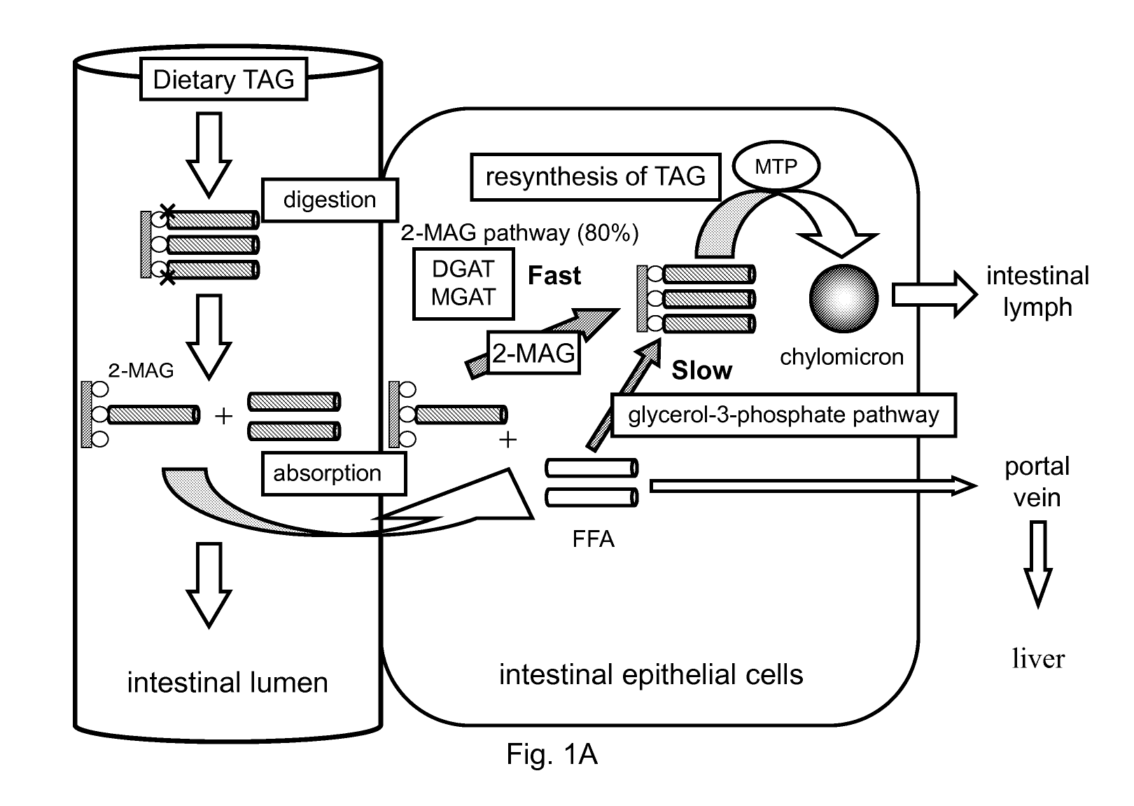

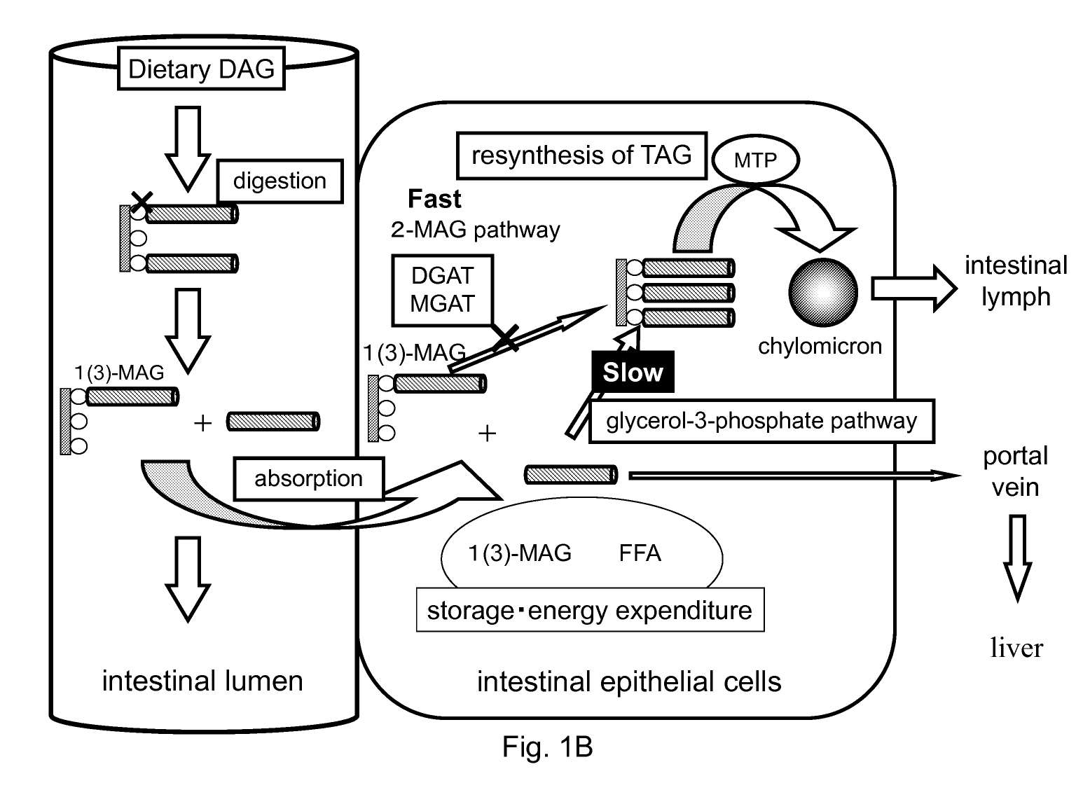

The DAG-rich fats and oils described herein provide an important source of energy, essential fatty acids and fat-soluble vitamins; while they impart an excellent flavor, texture, and palatability to food. Moreover, because of their structure and metabolic profile, they are beneficial for managing certain markers of metabolic syndromes such as postprandial hyperlipemia, insulin resistance, LDL and HDL blood levels. See Hidekatsu Yanai, Yoshiharu Tomono, Kumie Ito, Nobuyuki Furutani, Hiroshi Yoshida and Noro Tada, Diacylglycerol oil for the metabolic syndrome, Nutritional Journal 2007, 6:43. Yanai et al. have previously explained the differences between TAG and DAG metabolism. In contrast to TAGs, 1,3 and/or 1,2 DAGs do not completely reassemble after they are digested and are absorbed through the intestinal lumen. As illustrated in FIG. 1, following absorption, the free fatty acids are transferred to the liver and used as a source of energy.

Hence, Applicant has discovered a fat and oil composition that while comprised of significant amount of saturated fatty acids: (1) has a beneficial effect on metabolic syndrome and/or CVD; (2) takes advantage of the superior physical properties of the semi-solid palm and other fat and oil to replace trans fat-containing shortenings and other semi-solid fats; and (3) develops a new market segment by providing a palm oil-based composition that features a reduced saturated TAG content. A semi-solid fat is a fat composition that is semi-solid at room temperature (e.g., shortening).

In addition, palm kernel oil and coconut oil, among other tropical oils, are rich sources of medium-chain triacylglycerides (MCTs). MCTs can be modified into medium-chain diacylglycerides (MCDs). MCDs are diacylglycerides in which the acyl moieties have a carbon chain length ranging from 6 to 12. Like other diacylglycerides, MCDs would be expected to be metabolized for energy needs rather than contribute to adiposity when consumed. Applicants' DAG-rich palm and palm kernel-derived oil and fat compositions can be engineered to contain high levels of MCDs. The known effects of medium-chain lipids in triacylglycerides, in addition to the reduction in LDL accompanying the use of DAGs, will provide health benefits associated with the consumption of the compositions disclosed herein.

“Tropical plants” are coconut, cocoa, shea and palm plants. “Tropical oils,” as used herein, refers to oils derived from tropical plants. “Temperate plants” are all plants not defined herein as tropical plants. “Oils from temperate plants” and “temperate plant oil(s)” are oils from temperate plants. Non-limiting examples of temperate oils include sunflower oil, soybean oil, corn oil, and canola oils. In one embodiment, sunflower oil, soybean oil, corn oil, and canola oils having a saturated fatty acid content of at least 15% can be used. “Alga(e)” is construed in the broadest possible sense, to include both unicellular and multicellular photosynthetic organisms, including cyanobacteria.

In some embodiments, a tropical oil can be selected from the group consisting of palm oil, palm kernel oil, cocoa oil, shea oil, coconut oil, and mixtures thereof. For example, a tropical can include palm oil, palm kernel oil, or mixtures thereof. In some embodiments, palm oil can be selected from palm olein, palm stearine, fractionated palm olein, palm mid-fraction, and mixtures thereof. In some cases, palm kernel oil can be selected from palm kernel olein, palm kernel stearin, and mixtures thereof.

Natural palm oil comes from the fruit of the oil palm tree, a tropical species that originated in West Africa, but now several varieties are grown in many parts of the world. Palm and other tropical oils have useful properties for applications in place of trans fats. In addition to being relatively inexpensive, palm oil is semi-solid at room temperature, making it well-suited for baking and food production. However, there is a strong perception that its high saturated fat content (50% for palm oil, 80% for palm kernel oil) is undesirable at a time when health agencies in the US and Europe, in particular, are trying to educate consumers about the need to lower daily intake of saturated fats. Reformulated solid fats should not contain increased contents of SFA. A primary consideration in the food industry today is to count the sum of TFA and SFA as “cholesterol elevating.” Thus, a need exists for reformulated solid fats with desirable cooking properties, but with greater health benefits.

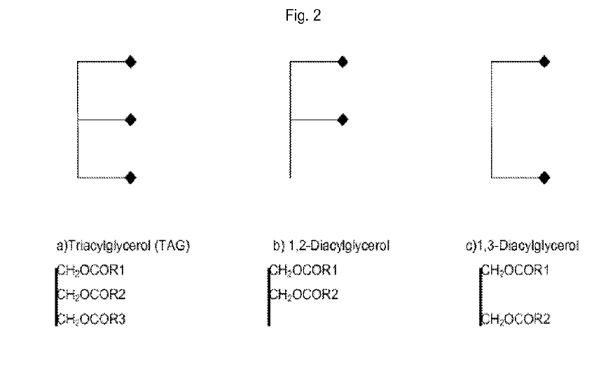

Natural palm oil is approximately 50% saturated fatty acid (SFA) (7 g in one tablespoon serving) and natural palm kernel oil is approximately 80% SFA (10 g in one tablespoon serving), with each having an approximate DAG content of 4 to 7.5%. Applicants' DAG-rich palm and palm kernel-derived oil and fat compositions described herein have higher DAG content than the parent oil, containing, in one embodiment, approximately 70% 1,3-DAGs and 30% 1,2-DAGs (see FIGS. 2B and 2C for chemical structures of 1,3-DAG and 1,2-DAG).

Fractions of palm and palm kernel oil can be obtained during refinement of crude palm oil. Fractionation of palm oil is typically accomplished by distillation or crystallization. In the case of both palm and palm kernel oil, the olein fraction is the liquid fraction obtained by either process. This fraction is a liquid at room temperature and typically contains primarily unsaturated fatty acids, including polyunsaturated fatty acids (PUFAs). The stearine fraction, on the other hand, is typically a solid at room temperature and is composed primarily of saturated fatty acids. Palm mid-fraction is a fat produced by multiple fractionations of palm oil. Its main characteristic is a very high concentration of symmetrical disaturated triglycerides (mainly 1,3-dipalmito-2-oleo triacylglycerol (POP)) resulting in a very steep solid fat content (SFC)/temperature curve.

The fatty acid composition of natural oils varies based on its plant source. For example, natural palm oil typically contains from about 0.1 to about 1.0% by weight 12:0 fatty acids (also known as lauric acid); from about 0.9 to about 1.5% by weight 14:0 fatty acids (also known as myristic acid); from about 40 to about 50% by weight (e.g., 41.8 to about 46.8 by weight) 16:0 fatty acids (also known as palmitic acid); from about 0.1 to about 1% by weight (e.g., about 0.1 to about 0.3% by weight) 16:1 fatty acids (also known as palmitoleic acid); from about 3 to about 6% by weight (e.g., about 4.2 to about 5.1% by weight) 18:0 fatty acids (also known as stearic acid); from about 30 to about 45% by weight (e.g., about 37.3 to about 40.8% by weight) 18:1 fatty acids (also known as oleic acid); from about 8 to about 12% by weight (e.g., about 9.1 to about 11.0% by weight) 18:2 fatty acids (also known as linoleic acid); from about 0 to about 0.6% by weight 18:3 fatty acids (also known as linolenic acid); and from about 0.2 to about 0.7% by weight 20:0 fatty acids (also known as archidic acid).

Natural palm kernel oil, on the other hand, typically contains from about 1 to about 4% by weight (e.g., about 3 to about 4% by weight) 8:0 fatty acids (also known as caprylic acid); from about 1 to about 7% by weight (e.g., about 3 to about 7% by weight 10:0 fatty acids (also known as capric acid); from about 45 to about 55% by weight (e.g., about 47 to about 52% by weight) 12:0 fatty acids; from about 14 to about 19% by weight (e.g., about 15 to about 17% by weight) 14:0 fatty acids; from about 5 to about 10% by weight (e.g, about 6 to about 9% by weight) 16:0 fatty acids; from about 1 to about 4% by weight (e.g., about 2 to about 3% by weight) 18:0 fatty acids; and from about 9 to about 20% by weight (e.g., about 10 to about 18% by weight) 18:1 fatty acids.

Provided herein is a semi-solid fat composition or an oil composition having from about 20 to about 100% by weight (e.g., about 25 to about 100% by weight; about 30 to about 100% by weight; about 35 to about 100% by weight; about 40 to about 100% by weight; about 45 to about 100% by weight; about 50 to about 100% by weight; about 55 to about 100% by weight; about 55 to about 100% by weight; about 60 to about 100% by weight; about 65 to about 100% by weight; about 70 to about 100% by weight; about 75 to about 100% by weight; about 80 to about 100% by weight; about 85 to about 100% by weight; about 90 to about 100% by weight; about 92 to about 100% by weight; about 94 to about 100% by weight; about 96 to about 100% by weight; about 60 to about 95% by weight; about 75 to about 95% by weight; about 75 to about 85% by weight; and about 85 to about 95% by weight) of diacylglycerols derived from a tropical oil or a temperate oil having a saturated fatty acid content of at least 15%. Non-limiting examples of suitable temperate oils include oils from high palmitic or stearic acid soybean, corn, sunflower, or canola lines. See, e.g., NUTRISUN™ high stearic, high oleic sunflower oil, HELIA high stearic, high oleic sunflower oil, and U.S. Pat. Nos. 5,750,846, 5,714,668 and 7,504,563.

In some embodiments, the fat or oil compositions have from about 70 to about 100% by weight DAG derived from a tropical oil (e.g., palm oil or palm kernel oil) or temperate oil having a saturated fatty acid content of at least 15%. For example, a composition can include about 75 to about 100% by weight; about 80 to about 100% by weight; about 85 to about 100% by weight; about 90 to about 100% by weight; about 92 to about 100% by weight; about 94 to about 100% by weight; about 96 to about 100% by weight; about 98 to about 100% by weight; about 75 to about 95% by weight; about 80 to about 90% by weight; and about 85 to about 90% by weight DAG. For example, the fat compositions can have about 89% by weight DAG.

The fat and oil compositions described herein can include at least about 25% by weight 1,3-DAG. For example, the compositions can include from about 50 to about 80% by weight 1,3-DAG (e.g., about 50 to about 78% by weight; about 50 to about 75% by weight; about 50 to about 72% by weight; about 50 to about 70% by weight; about 50 to about 68% by weight; about 50 to about 65% by weight; about 50 to about 62% by weight; about 50 to about 60% by weight; about 55 to about 80% by weight; about 58 to about 80% by weight; about 60 to about 80% by weight; about 65 to about 80% by weight; about 68 to about 80% by weight; about 72 to about 80% by weight; about 75 to about 80% by weight; about 52 to about 62% by weight; about 54 to about 60% by weight; about 56 to about 59% by weight; about 60 to about 75% by weight; about 68 to about 78% by weight; or about 67% to about 73% by weight 1,3-DAG). In some embodiments, the compositions can include >80% by weight 1,3-DAG. For example, the compositions can include about 80 to about 100% by weight 1,3-DAG such as about 80 to about 95%, about 80 to about 90%, about 82 to about 98%, about 85 to about 95% by weight 1,3-DAG.

In some embodiments, the compositions include at least 25% by weight 1,3-DAG and from about 15 to about 40% by weight 1,2-DAG (e.g., about 15 to about 25% by weight, about 15 to about 22% by weight; about 18 to about 25% by weight; about 20 to about 25% by weight; about 18 to about 22% by weight; about 19 to about 22% by weight, about 20 to about 40% by weight, about 25 to about 40% by weight, or about 30 to about 40% by weight 1,2-DAG). In some embodiments, a composition can include about 58% by weight 1,3-DAG and about 21% by weight 1,2-DAG. In some embodiments, the composition includes about 60% by weight 1,3-DAG and 40% by weight 1,2-DAG. In some embodiments, the composition includes about 50% by weight 1,3-DAG and 50% by weight 1,2-DAG.

The fatty acids present in such 1,2- and 1,3-DAGs can depend on the composition of the oil from which they are derived. For example, in some embodiments, the DAG include a saturated fatty acid having a chain length of 8-18 carbon atoms at either of the 1(3) or 2 positions, or at both the 1(3) and 2 positions. In some embodiments, the DAG include one or more unsaturated fatty acids, such as 18:1, 18:2, 18:3 (e.g., omega-3 and omega-6), 18:4, 20:3, 20:4, 20:5, and 22:6 (omega-6) fatty acids at either of the 1(3) or 2 positions, or at both the 1(3) and 2 positions. In some embodiments, the unsaturated fatty acid is gamma-linolenic acid or stearidonic acid (18:4). In some embodiments, the DAG contains an unsaturated fatty acid at the 2 position and a saturated fatty acid at the 1(3) position.

In some embodiments, a semi-solid fat or oil composition is provided that contains from about 20 to about 95% by weight of DAG, wherein the fatty acid moieties of the DAG are derived from a tropical oil and from one or more other oils. For example, the composition can comprise from about 25 to about 95% by weight; about 35 to about 95% by weight; about 45 to about 95% by weight; about 55 to about 95% by weight; about 65 to about 95% by weight; about 75 to about 95% by weight; about 85 to about 95% by weight; about 20 to about 90% by weight; about 20 to about 70% by weight; about 20 to about 60% by weight; about 20 to about 50% by weight and about 20 to about 40% by weight DAGs. For example, in one embodiment, the fatty acid moieties in the DAG can be derived from a tropical oil and from free fatty acids derived from one or more other oils. The free fatty acids can be selected from the group consisting of mono-unsaturated fatty acids (MUFAs), poly-unsaturated fatty acids, (PUFAs), medium-chain fatty acids, and combinations thereof. These fatty acids can be derived from one or more of fish, algae, tropical plants, and vegetables. For example, the free fatty acids can be derived from palm, palm kernel, coconut, sunflower, corn, soybean, or canola oils. In some embodiments, the fatty acids include one or more of an 18:1, 18:2, 18:3 (e.g., omega-3 and omega-6), 18:4, 20:3, 20:4, 20:5, and 22:6 (omega-6) fatty acids. For example, the free fatty acid can be gamma-linolenic acid or stearidonic acid.

Also provided herein is a fat composition consisting essentially of from about 60 to about 100% by weight of DAG. For example, a fat composition can consist essentially of from about 65 to about 100% by weight; from about 70 to about 100% by weight; from about 75 to about 100% by weight; from about 80 to about 100% by weight; from about 85 to about 100% by weight; from about 87 to about 100% by weight; from about 90 to about 100% by weight; from about 93 to about 100% by weight; from about 95 to about 100% by weight; from about 70 to about 95% by weight; from about 75 to about 90% by weight; from about 80 to about 95% by weight; and from about 80 to about 90% by weight of DAG. In some embodiments, the composition consists essentially of from about 85 to about 90% by weight of the DAG. In some embodiments, the composition consists essentially of from about 75 to about 85% by weight of the DAG.

The diacylglycerols in such compositions can include from about 60 to about 80% by weight 1,3-diacylglycerols and from about 20 to about 40% by weight 1,2-diacylglycerols.

In some embodiments, the fatty acid content of the DAG comprises from about 40 to about 50% by weight 16:0 fatty acid, from about 3 to about 6% by weight 18:0 fatty acid, from about 30 to about 45% by weight 18:1 fatty acid, and from about 7 to about 12% by weight 18:2 fatty acid. For example, the fatty acid content of the DAG can comprise about 46% by weight 16:0 fatty acid, about 4% by weight 18:0 fatty acid, about 36% by weight 18:1 fatty acid, and about 9% by weight 18:2 fatty acid.

In some embodiments, the fatty acid content of the DAG in such compositions comprises from about 45 to about 55% by weight 12:0 fatty acid; from about 15 to about 20% by weight 14:0 fatty acid; from about 6 to about 12% by weight 16:0 fatty acid; and from about 10 to about 20% by weight 18:1 fatty acid. For example, the fatty acid content of the DAG can comprise about 47% by weight 12:0 fatty acid; about 18% by weight 14:0 fatty acid; from about 9% by weight 16:0 fatty acid; from about 16% by weight 18:1 fatty acid.

Such fat compositions may have trace levels of monoacylglycerol. In some embodiments, the composition will have less than about 5% by weight monoacylglycerol (e.g., less than about 4% by weight, less than about 3% by weight, less than about 2% by weight, less than about 1% by weight, less than about 0.5% by weight, less than about 1% by weight). In some cases, the composition will lack a detectable level of monoacylglycerol.

In some embodiments, such fat compositions have from about 5 to about 25% by weight triacylglycerol. For example, less than about 25% by weight triacylglycerol, less than about 20% by weight triacylglycerol, or less than about 15% by weight triacylglyerol.

In some embodiments, the diacylglycerols of such compositions are derived from palm oil. In some embodiments, the diacylglycerols of such compositions derived from palm kernel oil.

The fat compositions described herein typically have a solid fat index of at least 15% at room temperature. For example, a fat composition can have a SFI ranging from about 15 to about 75% at room temperature (e.g., about 20 to about 60%; about 22 to about 50%; and about 25 to about 45%). SFI can be determined across a range of temperatures using a method based on AOCS Cd 10-57. These measurements describe the percentage of a product that is in a crystalline (solid) phase across a temperature gradient. The creation of this curve gives an understanding of properties and performance of oils over a range of temperatures, information that can be used to create basestocks for blending the fat composition to produce margarines or shortenings. SFI is determined using dilatometry, a technique that measures the changes in volume that occur when a solid goes to liquid. The measurements can be used with oils and fats with a SFI of 50 or less at 10° C.

In some embodiments, the fat and oil compositions described herein contain trace amounts of one or more of phytosterols, phytostanols, phytosterol esters, or phytostanol esters. For example, the compositions can include less than 1% by weight (e.g., less than about 0.5% by weight, less than about 1% by weight) of the phytosterols, phytostanols, phytosterol esters, phytostanol esters, or mixtures thereof. In some embodiments, the compositions lack a detectable amount of one or more of phytosterols, phytostanols, phytosterol esters, or phytostanol esters. Phytosterols and phytostanols can be selected from α-sitosterol, β-sitosterol, stigmasterol, ergosterol, campesterol, α-sitostanol, β-sitostanol, stigmastanol, and campestanol.

In some embodiments, the fat and oil compositions described herein contain trace amounts of one or more antioxidants. For example, the compositions can include less than 1% by weight (e.g., less than about 0.5% by weight, less than about 1% by weight) of one or more antioxidants. In some embodiments, the compositions lack a detectable amount of one or more antioxidants. Non-limiting examples of antioxidants include Vitamin E, butylhydroxytoluene, butylhydroxyanisole, tert-butylhydroquinone, Vitamin C, derivatives of Vitamin C, phospholipids, rosemary extract, gallic acid, catechin, Vitamin A, Vitamin E, and mixtures thereof.

In some embodiments, the fat and oil compositions described herein contain trace amounts of one or more compounds or extracts selected from ferulic acid esters, squalene, polyphenols; palmitate, tochopherols, tocotrienol; solid defatted milk; capsicum extract; green tea extract; Rosmarinus officinalis extract; and minerals such as copper, iron, manganese and cobalt. For example, the compositions can include less than 1% by weight (e.g., less than about 0.5% by weight, less than about 1% by weight) of one or more of such compounds or extracts. In some embodiments, the compositions lack a detectable amount of one or more of such compounds or extracts.

The fat (e.g., semi-solid fat compositions) and oil compositions described herein can be used in food compositions. Examples of such food compositions include cakes, pies, breads, muffins, crackers, sweet dough, pastry, cream filling, yogurt, pudding, cream cheese, spreads, salad dressing, margarines, butter, mayonnaise, frozen foods, ice cream, frozen desserts, frozen yogurt, smoothies, peanut butter, granola, bars, cookies, beverages, and medical foods such as meal replacement beverages and bars, and fluids used for enteric and tube feedings. In some embodiments, an ingredient in the food composition comprises a fat or oil composition as described herein. Non-limiting examples of such ingredients include shortening, bakery fat, frying fat, coating fats, non-dairy fats, cocoa-butter equivalent, butter, margarine, and vanaspati ghee.

Applicant's DAG-rich palm oil compositions have an improved shelf life and resistance to becoming stale. Incorporation of DAGs has been shown to improve emulsion stability, and can reduce the rate of formation of compounds that are associated with stale flavors. Incorporation of DAGs reduces water activity in starches and proteins, comprising the food matrix and, consequently, reduces processes leading to the formation of stale flavors. Accordingly, applications of the compositions disclosed herein include a wide variety of uses for which a healthier solid fat profile, improved shelf-life, and staling properties are sought. These include deep fat fries, shortening, and cocoa-butter substitutes in confectionary foods. Additionally, there are more expensive products for which applicant's DAG-rich palm-derived oil and fat compositions are a superior, healthier oil product, including, but not limited to, specialty fats used in confectionary, e.g., cocoa butter equivalent, cocoa butter substitutes, toffee fat, non-dairy fat (e.g., for use in ice-cream), cream filling fat, bakery fats (e.g., for use in desserts such as cakes, cheesecakes, pies, pastries, breads, etc) and general purpose coating fat. Some of these uses are detailed below.

Deep fat frying is an important food preparation and processing method and it is one that is nearly universally practiced. For deep-frying purposes, the oil or fat should have a low polyunsaturated fat (“PUFA”) profile, especially of linolenic acid, which tends to oxidize very rapidly. Commercial frying operations tend to use solid fats rather than liquid oils, primarily to minimize oxidation of the oils and to extend the shelf life of the fried products.

Shortenings, including bakery fats, are used extensively in the food industry. An important function of a shortening is its ability to incorporate and then hold air when beaten in a cake batter or creamed with sugar. The trapping of air facilitates the formation of a porous structure and increases the volume of the cream and the baked product. Shortenings also contribute to lubrication and give the dough the required final consistency. Such properties cannot be imparted by native liquid oils, which lack the appropriate solids content. Applicant's DAG-rich palm derived, or other DAG-rich, shortenings provide a healthy alternative due to its semisolid physical properties. Shortenings comprising DAG-rich palm oils preferably vary from 10-90%, more preferably 20-70%, still more preferably, 25-60%, and even more preferably from 30-40% DAG-rich palm oil. Applicant's DAG-rich palm oil compositions have an SFI of at least 15% (e.g., 22-25%) at room temperature, and this composition stabilizes the shortening and assists in good baking performance. Among the variety of trans-fat-free cake shortenings possible with DAG-rich palm-based products, are numerous specially designed shortenings for specific applications, such as layer and pound cakes, sweet dough, breads and cream fillers and are also excellent as pastry and bread fats.

The above-described foodstuffs, and others containing such DAG-rich ingredients can also be utilized to prepare frozen foods, such as, for example, ice cream, frozen desserts, frozen yogurt and similar products.

Provided herein is a food product having a fat or oil composition described herein. For example, a food product can include a fat or oil composition comprising from about 20 to about 100% by weight of diacylglycerols derived from a tropical oil (e.g., palm or palm kernel oil) or a temperate oil (e.g., a temperate oil having a saturated fatty acid content of at least 15%). In some embodiments, the fat in the food product can consist essentially of such fat or oil compositions. Non-limiting examples of food products include cakes, pies, breads, muffins, crackers, sweet dough, pastry, cream filling, yogurt, pudding, cream cheese, spreads, salad dressing, margarines, butter, mayonnaise, frozen foods, ice cream, frozen desserts, frozen yogurt, smoothies, peanut butter, granola, bars, and cookies. An existing food product can be improved by substituting a fat or oil composition comprising diacylglycerols as described herein for about 5 to about 100% of a hydrogenated fat or oil in the food product.

Margarines are defined as liquid or plastic emulsions containing 80% or more fat, not more than 16% water, and generally fortified with vitamin A. There are several types of margarines, each formulated to fulfill a specific requirement. Applicant's DAG-rich palm-derived oil and fat compositions provide for a superior, healthier margarine than their natural counterparts or the TFA-rich margarines to be replaced. Such DAG-rich compositions provide good physical properties necessary for quality margarines, including emulsion stability without undue oil separation, reduced brittleness, good spreadability, and a clean, smooth melt in the mouth capability.

Vegetable ghee, or vanaspati, is a major dietary fat source in many developing countries of the Middle East, Indian sub-continent, Afghanistan and South-East Asia. Differences in regional preferences of vanaspati are amplified by the texture of the product ranging from completely smooth to granular, depending on specific culinary practices. Vanaspati is traditionally produced with a range of fat blends, including a very high level of TFA containing hydrogenated fats. Applicant's DAG-rich palm-derived oil and fat compositions may be incorporated as a base ingredient (up to 100%), or as a blend with various soft oils.

Compositions disclosed herein can be used in many common household foodstuffs to improve shelf-life, flavor, consistency or beneficial health properties. As non-limiting examples, such a composition can be incorporated into peanut butter, cream cheese, yogurt and/or cookies.

In some embodiments, the fat and oil compositions described herein can be used to produce a food product. For example, a food product can be prepared by substituting from about 5 to about 100% (e.g., about 10 to about 100%; about 15 to about 100%; about 20 to about 100%; about 25 to about 100%; about 30 to about 100%; about 35 to about 100%; about 40 to about 100%; about 45 to about 100%; about 50 to about 100%; about 60 to about 100%; about 75 to about 100%; about 85 to about 100%; about 90 to about 100%; about 5 to about 95%; about 5 to about 90%; about 5 to about 80%; about 5 to about 70%; about 5 to about 60%; about 5 to about 50%; about 5 to about 40%; about to about 30%; about 5 to about 15%; about 10 to about 80%; about 20 to about 70%; about 25 to about 75%; about 35 to about 65%; and about 25 to about 50%) of a trans fatty acid or saturated fatty acid in the food product with diacylglycerols derived from a tropical oil (e.g., a palm oil, palm kernel oil, or a mixture thereof) or a temperate oil (e.g., a temperate oil having a saturated fatty acid content of at least 15%). In some cases, the food product is improved by substituting a fat or oil composition as described herein for a percentage of a hydrogenated fat or oil in the food product. For example, a composition described herein can be substituted for about 5 to about 100% of a hydrogenated fat or oil (e.g., about 10 to about 100%; about 20 to about 100%; about 40 to about 100%; about 60 to about 100%; about 80 to about 100%; about 5 to about 65%; about 5 to about 45%; and about 5 to about 25%).

The fat and oil compositions described herein, and the food compositions and products containing the fat and oil compositions, can exhibit beneficial health effects when ingested by a mammal. Non-limiting examples of such health benefits include, for example, lowered serum LDL, raised serum HDL, and lowered total cholesterol.

For example, provided herein is a method for lowering total serum cholesterol and/or lowering serum LDL in a mammal comprising administering to the mammal an amount of a composition described herein to provide at least about 15 g/day (e.g., at least about 30 g/day or at least about 40 g/day) of diacylglycerols (e.g., derived from a tropical or temperate oil). In some embodiments, from about 15 g/day to about 300 g/day of the diacylglycerols are provided to the mammal (e.g., about 15 g/day to about 250 g/day; about 15 g/day to about 200 g/day; about 15 g/day to about 150 g/day; about 15 g/day to about 125 g/day; about 15 g/day to about 100 g/day; about 15 g/day to about 90 g/day; about 15 g/day to about 80 g/day; about 15 g/day to about 75 g/day; about 15 g/day to about 60 g/day; about 15 g/day to about 50 g/day; about 15 g/day to about 45 g/day; about 15 g/day to about 30 g/day; about 20 g/day to about 60 g/day; about 25 g/day to about 50 g/day; about 30 g/day to about 45 g/day; about 30 g/day to about 100 g/day; about 40 g/day to about 80 g/day; about 40 g/day to about 75 g/day).

Lowering of total serum cholesterol and/or serum LDL typically requires administration of a composition described herein for a continuous period of time. In some embodiments, the lowering of total serum cholesterol and/or serum LDL occurs after administering the composition for at least four weeks (e.g., at least 8 weeks; at least ten weeks; at least twelve weeks, fourteen weeks; at least twenty weeks; or at least 24 weeks).

Mammals include, for example, humans; non human primates, e.g. apes and monkeys; cattle; horses; sheep; rats; mice; pigs; and goats. In some embodiments, a mammal is a human.

In some cases, the mammal has been diagnosed as having a cardiovascular disorder (e.g., hypertriglyceridemia, hypercholesterolemia, other hyperlipidemias, hyperglycemia, hyperinsulinemia, arteriosclerosis, atherosclerosis, arteriolosclerosis, angina pectoris, thrombosis, myocardial infarction, and hypertension). In some embodiments, the mammal has been diagnosed as having type II diabetes. In some embodiments, the mammal has been diagnosed as having a metabolic syndrome. For example, the mammal may have a serum level of LDL ranging from about 120 to about 175 mg/dL prior to administration.

The compositions, as described herein, may be administered in a variety of forms, e.g., a tablet, pill, capsule, elixir, wafer, beverage, or consumed through ingestion of a food product comprising a composition having diacylglycerols as described herein.

### Production of DAG

Without limiting as the variety of methods of production available, in the present invention, palm oil, palm kernel oil, coconut oil as well as combinations with other oils, including but not limited to sunflower, corn, soybean, etc, can be modified into diacylglycerols (“DAG”) by, e.g., the removal of one of the fatty acids on the glycerol backbone of the triacylglyceride parent oil or by the direct synthesis of diacylglycerol molecules. Diacylglycerols can be synthesized by a variety of methods, including by enzymatic or non-enzymatic means; for example, DAG production can be achieved using lipases. See, e.g., Janni Brogaard Kristensen, Xuebing Xu and Huiling Mu, Diacylglycerol synthesis by enzymatic glycerolysis: Screening of commercially available lipases, J. Amer. Oil Chemists' Society, Vol. 82, No. 5, 2005, p. 329-334. For the production of DAG for an industrial scale, the reuse of the enzyme is advantageous and can be accomplished in a number of ways. Generally, an enzyme can be stabilized by immobilization.

Both the yield of 1,3-DAG and the purity of DAG can be optimized by variations in experimental conditions, including reaction temperature, pressure, and amount of enzyme present. An increase in temperature or the amount of enzyme used can result in an increase in the 1,3-DAG production rate. Vacuum is important for attaining high yields of 1,3-DAG. Under conditions of a high vacuum (1 mm Hg) at 50° C., 1.09 M 1,3-DAG can be produced from 1.29 M glycerol and 2.59 MFA in an 84% yield and in 90% purity (T. Watanabe, et al., J. Amer. Oil Chemists Society, 2003 80(12):1201-1207). For the lipase-catalyzed synthesis of 1,3-DAG, the presence of n-hexane is preferred for the maintenance of lipase activity. In one embodiment of the present invention, the optimum yield (40%) of 1,3-DAG synthesis can be obtained when the reaction is carried out with n-hexane/octane (1:1, v/v) (H. F. Liao, et al., Biotechnology Letters 2003 25 (21): 1857-1861).

Biocatalysed synthesis of sn-1,3-diacylglycerol oil from palm oil, palm kernel oil or other tropical or temperate oils, or mixtures thereof, performed in two major steps, without isolation of the intermediates, can be carried out. Ethanolysis of palm oil, palm kernel oil, or potentially, other tropical oils, using immobilized non-regiospecific lipase from Candida antarctica (Novozym 435) can be carried out to obtain glycerol (Gly) and fatty acid ethyl esters (FAEE). In a second step the ethanolysis products can be re-esterified using different sn-1,3-regiospecific lipases, both immobilized and non-immobilized, in different reaction media, that is in the presence of solvents or in a solvent-free system, for different times, at different temperatures (12, 25 and 40° C.). The lipase from Rhizomucor miehei (Lipozyme™) has been the most effective among the sn-1,3-specific lipases screened. (F. Blasi, et al. Enzyme and Microbial Tech. 2007 41 (6-7): 727-732.)

In some embodiments, preparation of DAGs can proceed by one of or a combination of the following methods. In some cases, DAGs can be prepared by esterification of the fatty acids with glycerol, see, for example, U.S. Pat. No. 7,709,667; and U.S. Publication No. 2010/0092650. In some cases, DAGs can be prepared by glycerolysis of triglycerides, for example, using alkali metal salts or alkali earth metal salts to drive glycerolysis. See, for example, U.S. Pat. No. 7,081,542. In some embodiments, DAGs can be prepared by reacting TAGs with water and an immobilized lipase, followed by water removal and separation of undesired products such as MAGs, TAGS, etc. See, for example, U.S. Publication No. 2008/0312342. In some cases, DAGs can be prepared by short-path distillation. For example, see U.S. Pat. No. 7,531,678.

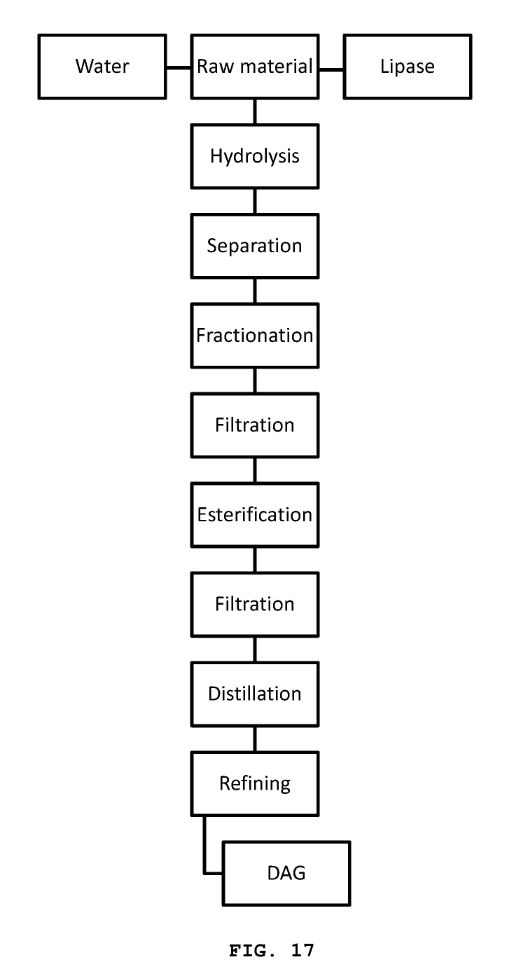

In some embodiments, DAGs can be prepared and purified using the method illustrated in FIG. 17. A fat or oil composition as provided herein can be prepared by using the following method where a lipase-based hydrolysis reaction is carried out with water and oil (e.g., palm oil, palm kernel oil, or a fraction or combination thereof) to produce glycerol and free fatty acids (FFA). The glycerol is separated from the FFA which are further fractionated and selectively isolated by filtration. The glycerol and isolated FFA are combined into DAGs by esterification with one or more sn-1,3-regiospecific lipases, for example, Rhizomucor miehei (Lipozyme RM IM™). The method further comprises purification (e.g., distillation and refining) to remove any un-reacted fatty acids, and glycerol to arrive at the final desired DAG product mixture.

### EXAMPLES

Aspects of the present teachings may be further understood in light of the following examples, which should not be construed as limiting the scope of the present teachings in any way.

In an embodiment, diacylglycerol(s), predominantly 1,3 diacylglycerol(s) and 1,2 diacylglycerol(s), are administered in combination with other liquid DAG oils and/or solid fats to create favorable metabolic and/or cardiovascular benefits and/or management of postprandial and fasting blood lipid levels.

In an embodiment, semi-solid diacylglycerol(s) DAG, predominantly 1,3 diacylglycerol(s) and 1,2 diacylglycerol(s), are administered in combination with other liquid DAG oils and/or fats with high stearic acid content, including but not limited to sunflower, corn, soybean, rapeseed, etc., and/or high palmitic content to create favorable metabolic and/or cardiovascular benefits and/or management of postprandial and fasting blood lipid levels.

In another embodiment, diacylglycerol(s) (DAG), predominantly 1,3-diacylglycerol(s), and phytosterol and/or phytostanol ester(s) combinations, or medium-chain triglycerides, are provided.

Another embodiment of the present invention, the composition of matter preferably comprises from 1 to 99 wt % diacylglycerol(s) and from 1 to 99 wt % phytosterol and/or phytostanol ester(s) dissolved or dispersed in edible oil and/or edible fat, and may further optionally comprise monoglycerides.

Another embodiment of the present invention provides compositions comprising combinations of diacylglycerol(s), predominantly 1,3-diacylglycerol(s), derived from palm oil and palm kernel oil and potentially other tropical oils, in combination with phytosterol and/or phytostanol ester(s) (PSE), dissolved or dispersed in edible oil and/or edible fat, in the manufacture of nutritional supplements and orally administrable pharmaceutical preparations or non-dispersed in an additional edible fat.

The phytosterol ester(s) in these compositions may be any fatty acid esters, for example but not limited to oleic and palmitic esters of stigmasterol, sitosterol, betasitosterol, brassicasterol, campesterol, 5-avenasterol and isomers and derivatives thereof.

In one embodiment of the present invention, a composition comprises a molar ratio between diacylglycerol(s) and phytosterol and/or phytostanol ester(s), from about 1:5 to about 5:1. In a particular embodiment, the amount of diacylglycerol(s) in a composition is from 1 to 99 wt %, preferably from 7 to 48 wt %, and the amount of phytosterol and/or phytostanol ester(s) in a composition is from 1 to 99 wt %, preferably from 5 to 50 wt %.

In another embodiment of the present invention, a composition consists of 15 wt % DAG, mainly 1,3-diacylglycerol(s) and 25 wt % total PSE dissolved or dispersed in an edible oil. In a particular embodiment, a composition can consist of 15 wt % DAG, mainly 1,3-diacylglycerol(s) and 25 wt % total phytosterol ester(s) (PSE) dissolved or dispersed in an edible oil.

In a pharmaceutical composition of the invention, the molar ratio between diacylglycerol(s) and phytosterol and/or phytostanol, ester(s) is preferably from about 1:5 to about 5:1. For example, the ratio can be from about 2:1 to about 5:1. In one embodiment, the amount of diacylglycerol(s) in a combination is at least 1 wt %. Further, in the pharmaceutical composition, the amount of phytosterol and/or phytostanol ester(s) in a combination is preferably at least 1 wt %.

In particular embodiments, the combination comprised in the pharmaceutical composition of the invention, consists of diacylglycerol(s) in an amount of from 1 to 99 wt %, preferably from 7 to 48 wt %, and the amount of phytosterol and/or phytostanol ester(s) in said combination is from 1 to 99 wt %, preferably from 5 to 50 wt %.

In other particular embodiments, the pharmaceutical composition of the invention consists substantially of 15 wt % DAG, mainly 1,3-diacylglycerol(s) and 25 wt % total PSE dissolved or dispersed in olive oil.

The diacylglycerol(s) may be obtained by any conventional enzymatic or non-enzymatic procedure. They may be obtained by inter-esterification reaction between phytosterol (s) and triglyceride(s) present in the oil and/or fat. The phytosterol and/or phytostanol ester(s) may be obtained by any conventional enzymatic or non-enzymatic procedure.

In particular embodiments, the composition of matter according to the invention comprises 1 to 99 wt % diacyglycerols, from 1 to 99 wt % phytosterol and/or phytostanol esters and from 0 to 50 wt % monoglycerides and from 1 to 99 wt % triacyglycerol(s) and from 1 to 99 wt % medium-chain triglycerides. More particularly, the composition of matter according to the invention comprises from 3 to 50 wt % diacyglycerols, from 7 to 48 wt % phytosterol and/or phytostanol esters and from 2 to 90 wt % triacyglycerol(s)

### Example 1

Compositions of the present invention can be used in many common household products to improve shelf-life, flavor, consistency, mouth feel, other sensory attributes or beneficial health properties of low fat and fat-free foods. The following non-limiting examples demonstrate that a Palm DAG composition of the present invention, referred to herein as “Heartlite” in Examples 1-3, can be incorporated into such food stuffs as peanut butter, cream cheese, yogurt, bakery products, granola bars and cookies as described herein.

Peanut Butter

| Irgredients | Amount | |
|---|---|---|
| Peanut Butter | 36 | g |
| Heartlite | 5 | g |
| Total | 41 | g |

| | |
|---|---|
| Yield | 1 Serving |

### Instructions:

1. Place Peanut Butter in the bowl of a stand mixer fitted with a paddle.
2. Place Heartlite in a microwave safe bowl and melt until liquid.
3. Remove Heartlite from microwave and add to peanut butter.
4. Mix on low speed until peanut butter and Heartlite are completely incorporated.
5. Be sure to scrape the sides of the bowl periodically to be sure the mixture is uniform.

Cream Cheese

| Irgredients | Amount | |
|---|---|---|
| Cream Cheese | 31 | g |
| Heartlite | 7 | g |
| Total | 38 | g |

| | |
|---|---|
| Yield | 1 Serving |

### Instructions:

1. Remove lid and foil from cream cheese container.
2. Place cream cheese in microwave for 15 seconds to warm slightly.
3. Weigh cream cheese in bowl of a stand mixer fitted with a paddle.
4. Place measured Heartlite in a microwavable bowl and heat in microwave until liquid.
5. Stir Heartlite with a fork until it returns to its white color and begins to solidify.
6. While mixer is running on slow speed slowly add Heartlite to cream cheese.
7. Continue to mix until cream cheese mixture is uniform and Heartlite has cooled.

Butter

| Irgredients | Amount | |
|---|---|---|
| Butter | 3 | g |
| Heartlite | 3 | g |
| Total | 6 | g |

| | |
|---|---|
| Yield | 1 Serving |

### Instructions:

1. Soften butter until room temperature. Place butter in the bowl of a stand mixer fitted with a paddle.
2. Place Heartlite in a microwave safe bowl and melt until liquid.
3. Remove Heartlite from microwave and stir until it becomes the consistency of whipped frosting.
4. Add the Heartlite to the softened butter and mix on low speed until creamed.
5. Be sure to scrape the sides of the bowl periodically to be sure the mixture is uniform.

Yogurt

| Irgredients | Amount | |
|---|---|---|
| Yogurt | 170 | g |
| Heartlite | 7 | g |
| Total | 177 | g |

| | |
|---|---|
| Yield | 1 Serving |

### Instructions:

1. Place the measured yogurt in a blender and blend on low speed just long enough to be sure yogurt is circulating well.
2. Place Heartlite in a microwavable dish and microwave until liquid.
3. Remove Heartlite from microwave and allow to come up to temperature slightly. Do not let the Heartlite solidify.
4. Stream melted Heartlite into blender while mixing on high speed.
5. After all of the Heartlite has been incorporated be sure to scrape the sides and lid of the blender. Return blender to high speed and mix another 45 seconds.

### Chocolate Chip Cookie

Recipe adapted from The Bakers' Manual

### Revised Third Edition

### By: Joseph Amendola

| | Original Recipe | | | A-0% | B-0% | C-0% | D-100% |
|---|---|---|---|---|---|---|---|
| | Baking | | | Heartlite | Heartlite | Heartlite | Heartlite |
| Ingredient | Measurement | | Grams | Grams | Grams | Grams | Grams |
| Unsalted butter | 1 lb 8 oz | | 680.4 | 170.0 | 170.0 | 42.5 | 0.0 |
| Heartlite | n/a | | n/a | 0.0 | 0.0 | 127.6 | 170.0 |
| Granulated Sugar | 12 | oz | 340.2 | 85.1 | 85.1 | 85.1 | 85.1 |
| Light Brown Sugar | 12 | oz | 340.2 | 85.1 | 85.1 | 85.1 | 85.1 |
| Egg whites | 8 | oz | 226.8 | 56.7 | 56.7 | 56.7 | 56.7 |
| Butter extract | n/a | | n/a | 0 | 4 | 4 | 4 |
| Molasses | n/a | | n/a | 0 | 16 | 16 | 16 |
| Vanillin | To taste | | 14.2 | 14 | 14 | 14 | 14 |
| Water | 1 | oz | 28.35 | 7.1 | 7.1 | 7.1 | 7.1 |
| Baking Soda | 0.5 | oz | 14.2 | 3.6 | 3.6 | 3.6 | 3.6 |
| Salt | 0.5 | oz | 14.2 | 3.6 | 3.6 | 3.6 | 3.6 |
| Pastry Flour | 2 | lb | 907.2 | 226.8 | 226.8 | 226.8 | 226.8 |
| Pear Puree | 2 | lb | n/a | 0 | 28.4 | 28.4 | 28.4 |
| Chocolate Chips | 1 | lb | 453.6 | 113.4 | 113.4 | 113.4 | 113.4 |

| | | | | | |
|---|---|---|---|---|---|
| Total | 3019.4 | 765.4 | 813.8 | 813.9 | 813.8 |

### Oven: Convection-Fan on Low-Preheated to 300° F. Instructions:

1. Place the sugar, butter, molasses, and salt in a mixing bowl. Using a stand mixer fitted with a paddle, cream ingredients until light and fluffy.
2. Melt Heartlite in microwave until liquid. Remove from microwave and stir constantly until Heartlite becomes the consistency of softened butter.
3. Dissolve baking soda in water. Set aside.
4. Slowly stream the eggs, and water and baking soda into the creamed butter mixture. After liquid is incorporated stop machine and scrape bowl. Return to low speed and mix for 30 more seconds.
5. Sift flour and vannilin.
6. Stop mixer, add flour, chocolate chips, and vannilin. Mix on low just until combined.
7. Bake for 8-10 minutes rotating cookies after 4 minutes.

### Cake Recipe: Test of Recipe Including Buttermilk to Increase Richness

Recipe from The Professional Chef

### Seventh Edition

### Culinary Institute of America

| | Original | | | |
|---|---|---|---|---|
| | Recipe | A-0% | B-75% | C-100% |
| | Baking | Heartlite | Heartlite | Heartlite |
| Ingredient | Measurement | Grams | Grams | Grams |
| Heartlite | 0 | 0.00 | 86.25 | 115.00 |
| Cocoa Powder | 40 | 40 | 40 | 40 |
| Baking Soda | 1.5 | 1.5 | 1.5 | 1.5 |
| Salt | 1.7 | 1.7 | 1.7 | 1.7 |
| Buttermilk | 115 | 115 | 28.75 | 0.00 |
| Sugar | 200 | 200 | 200 | 200 |
| Light Brown | 115 | 115 | 115 | 115 |
| Sugar | | | | |
| Vanillin | 15 | 15 | 15 | 15 |
| Egg whites | 110 | 110 | 110 | 110 |
| Buttermilk | 226.8 | 226.8 | 226.8 | 226.8 |
| Pear Puree | n/a | 56.7 | 56.7 | 56.7 |
| Cake Flour | 140 | 140 | 140 | 140 |
| Total | 965 | 1021.7 | 1021.7 | 1021.7 |

### Oven: Convection-Fan on Low-Preheated to 300° F. Instructions

1. Place sugars, salt, and softened butter in bowl of stand mixer fitted with a paddle.
2. Cream sugar mixture on medium speed until light and fluffy.
*3. Melt Heartlite in microwave until completely liquid. Remove from microwave and stir constantly until Heartlite becomes the texture of whipped frosting. * Directions for samples including Heartlite only
4. Add Heartlite to creamed butter/sugar mixture and continue mixing until Heartlite is incorporated and mixture is again light and fluffy.
6. While mixer is on low speed slowly add eggs, pear puree, and buttermilk. Scrape the bowl and mix again making sure the batter is uniform.
7. Sift vanillin, flour and baking soda.
8. Add sifted flour mixture to batter and stir just until combined.
9. Fill cupcake tins (lined with parchment liners) ⅔ full and bake at 300° F. for 12-15 minutes or until done.
Notes: Batter was portioned into cupcake tins lined with parchment liners and baked for 15 minutes. Cupcake tins were rotated half way through baking. Each sample was baked individually.

### Carrot Muffin

Recipe adapted from The Professional Chef

### Revised Seventh Edition

### By Culinary Institute of America

| | Original | A-0% | B-75% | C-100% |
|---|---|---|---|---|
| | Recipe | Heartlite | Heartlite | Heartlite |
| Ingredient | Grams | Grams | Grams | Grams |
| Cake Flour | 317 | 79.3 | 79.3 | 79.3 |
| Whole Wheat | 317 | 79.3 | 79.3 | 79.3 |
| Flour | | | | |
| Heartlite | n/a | 0.00 | 55.1 | 73.5 |
| Rice Krispies | 33 | 8.3 | 8.3 | 8.3 |
| Granulated | 455 | 113.3 | 113.3 | 113.3 |
| Sugar | | | | |
| Baking Soda | 24 | 6.0 | 6.0 | 6.0 |
| Cinnamon, | 9 | 2.3 | 2.3 | 2.3 |
| Ground | | | | |
| Salt | 8 | 2.0 | 2.0 | 2.0 |
| Cloves, Ground | 1 | 0.3 | 0.3 | 0.3 |
| Apples, Grated | 686 | 171.5 | 171.5 | 171.5 |
| Carrots, Grated | 117 | 29.3 | 29.3 | 29.3 |
| Vegetable Oil | 294 | 73.5 | 73.5 | 73.5 |
| 2% Milk | 116 | 29.0 | 29.0 | 29.0 |
| Pure Vanilla | 18 | 4.5 | 4.5 | 4.5 |
| Extract | | | | |
| Egg Whites | 153 | 38.3 | 38.3 | 38.3 |
| Total | 2548 | 636.9 | 636.9 | 636.9 |

### Oven: Convection-Fan on Low-Preheated to 325° F. Instructions:

1. Peel, core and quarter apples. Peel carrots.
2. Using a robocoup fitted with a shredder attachment, shred apples and carrots. Set aside.
3. Sift flours, cinnamon, cloves, and baking soda into a large mixing bowl. Add Rice Krispies to sifted ingredients.
4. Place sugar, salt, and oil in bowl of a stand mixer fitted with a paddle.
*5. Melt Heartlite in microwave until liquid. Constantly stir the Heartlite until the fat solidifies and becomes the consistency of whipped frosting.
6. Cream the Heartlite, sugar, oil and salt.
7. Slowly add egg and vanilla to creamed Heartlite. Be sure to scrape sides of the bowl as necessary.
8. Add shredded apples and carrots to egg mixture.
9. Stir in milk until incorporated.
10. Add sifted ingredients and stir just until incorporated.
11. Portion 2 oz of batter into muffin tins lined with paper liners and bake for 15 minutes or until done.

### Granola Bar

Recipe adapted from The Breakfast Book

### By: Marion Cunningham

| | Original | A-0% | B-75% | C-100% |
|---|---|---|---|---|
| | Recipe | Heartlite | Heartlite | Heartlite |
| Ingredient | Grams | Grams | Grams | Grams |
| Shortening | 127.8 | 127.8 | 32.9 | 0.0 |
| Light Brown | 24.0 | 24.0 | 24.0 | 24.0 |
| Sugar | | | | |
| Granulated | 68 | 68.0 | 68.0 | 68.0 |
| Sugar | | | | |
| Strong Coffee | 56.7 | 56.7 | 56.7 | 56.7 |
| Egg whites | 60 | 60.0 | 60.0 | 60.0 |
| Rolled Oats | 205 | 205.0 | 205.0 | 205.0 |
| AP Flour | 130 | 130.0 | 130.0 | 130.0 |
| Salt | 6 | 6.0 | 6.0 | 6.0 |
| Baking Soda | 2.5 | 2.5 | 2.5 | 2.5 |
| All-Bran | 93.0 | 93.0 | 93.0 | 93.0 |
| Cereal | | | | |
| Heartlite | n/a | 0.0 | 95.9 | 127.8 |
| Total | 773.0 | 773.0 | 678.1 | 773.0 |

### Oven: Convection-Fan on Low-Preheated to 300° F.

### Instructions:

1. Place shortening, sugars and salt in a bowl of a stand mixer fitted with a paddle and mix until smooth and blended.
*2. Melt Heartlite in microwave until liquid. Constantly stir the Heartlite until the fat solidifies and becomes the texture of whipped frosting.
3. Add Heartlite to shortening mixture and cream until uniformly mixed.
4. Sift flour and baking soda into a medium mixing bowl. Add oats and All-Bran. Be sure all ingredients are well incorporated.
5. Slightly beat eggs in a small mixing bowl.
6. Slowly add coffee and eggs to creamed butter mixture. Be sure to scrape the sides of the bowl until all ingredients are uniformly incorporated.
7. Add dry ingredients and stir until just incorporated.
8. Grease and flour 3 half hotel pans.
9. Press batter onto prepared pans.
10. Bake for 10 minutes, rotate pans, and return to oven for 10 more minutes or until done.

### Granola Bar

Recipe adapted from The Breakfast Book

### By: Marion Cunningham

| | Original | A-0% | B-75% | C-100% |
|---|---|---|---|---|
| | Recipe | Heartlite | Heartlite | Heartlite |
| Ingredient | Grams | Grams | Grams | Grams |
| Shortening | 127.8 | 127.8 | 32.9 | 0.0 |
| Light Brown | 24.0 | 24.0 | 24.0 | 24.0 |
| Sugar | | | | |
| Granulated | 68 | 68.0 | 68.0 | 68.0 |
| Sugar | | | | |
| Strong Coffee | 56.7 | 56.7 | 56.7 | 56.7 |
| Egg whites | 60 | 60.0 | 60.0 | 60.0 |
| Rolled Oats | 205 | 205.0 | 205.0 | 205.0 |
| AP Flour | 130 | 130.0 | 130.0 | 130.0 |
| Salt | 6 | 6.0 | 6.0 | 6.0 |
| Baking Soda | 2.5 | 2.5 | 2.5 | 2.5 |
| All-Bran Cereal | 93.0 | 93.0 | 93.0 | 93.0 |
| Heartlite | n/a | 0.0 | 95.9 | 127.8 |
| Total | 773.0 | 773.0 | 678.1 | 773.0 |

### Oven: Convection-Fan on Low-Preheated to 300° F.

### Instructions:

1. Place shortening, sugars and salt in a bowl of a stand mixer fitted with a paddle and mix until smooth and blended.
*2. Melt Heartlite in microwave until liquid. Constantly stir the Heartlite until the fat solidifies and becomes the texture of whipped frosting.
3. Add Heartlite to shortening mixture and cream until uniformly mixed.
4. Sift flour and baking soda into a medium mixing bowl. Add oats and All-Bran. Be sure all ingredients are well incorporated.
5. Slightly beat eggs in a small mixing bowl.
6. Slowly add coffee and eggs to creamed butter mixture. Be sure to scrape the sides of the bowl until all ingredients are uniformly incorporated.
7. Add dry ingredients and stir until just incorporated.
8. Grease and flour 3 half hotel pans.
9. Press batter onto prepared pans.
10. Bake for 10 minutes, rotate pans, and return to oven for 10 more minutes or until done.

### Example 2

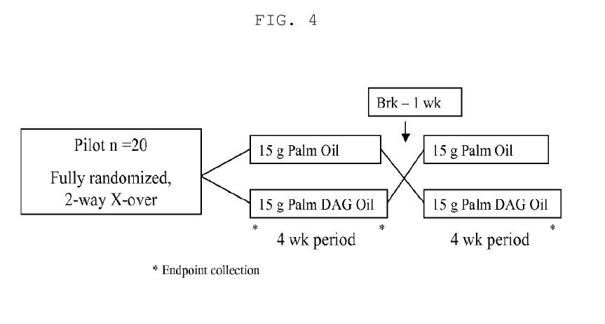

The inventors determine whether a diet that includes 15 g/day of the palm or palm kernel DAG fat improves the lipid and lipoprotein profile in moderately hypercholesterolemic individuals when compared to the parent fat (palm or palm kernel) (FIG. 4).

Individuals (n=20) with moderately elevated or elevated (see below) LDL cholesterol and triglycerides are recruited for a controlled feeding study. The study is a randomized, 2-period, blinded cross-over design (see diagram below). During the entire study both groups eat a control background diet and all foods are provided for the feeding periods. During each treatment period of 4 weeks, the different fats will be incorporated into recipes (i.e. spreads, peanut butter, cream cheese) according to the diet group, Palm Oil (PO) or Palm Oil DAG (POD). Participants have blood drawn and weight and blood pressure (BP) checked at the beginning of the study and at the end of each diet period, on two consecutive days. If there is a break of more than 2 weeks before the start of the second diet period, an additional blood draw is done to establish a baseline. Samples are assayed for lipid profile with aliquots reserved for additional assays (inflammatory markers) if determined to be appropriate.

Participants are healthy men and women, 30-60 years of age, with moderately elevated LDL-C (120-175 mg/dL) or elevated LDL-C (>175 img/dL) and with HDL-C of 30-50 mg/dL and triglycerides of 120-350 mg/dL. For this study, participants who, by Harris-Benedict equation, will require a total calorie level/day of 2100-3000 are selected. This will allow for one dose of the test fat at 15-20 g for all participants. Subjects are excluded if they are smokers, have diabetes, are pregnant or expecting to be pregnant, or lactating in the last 6 months. Those people who are taking cholesterol-lowering medications, including statins (although it is recognized that statins would not affect the outcome for a particular person) are excluded. Blood pressure lowering medications are acceptable if the person has controlled BP, <140/90 mmHg.

Diet Design: The background control diet is designed to meet current dietary recommendations—high in fruits and vegetables, whole grains, low-fat dairy, and lean meats. The macronutrient profile is: 25-32% total fat, 15-18% protein, ˜55% CHO, with 10 g/1000 kcal fiber/day and dietary cholesterol <300 mg/day. The test diets provide <10% of calories from saturated fat from all sources, including the test fats. The 15 g test fat dose is set for the 2100-2400 kcal level. For the 15 g PO or POD diet, 15 of the POD fat for 15 g of the parent fat is substituted. This approach controls for all other sources of fat so that the effects of DAG fat vs. the parent fat are tested specifically. Each day, with meals or as part of a snack, the participant has DAG or parent fat-containing products to eat—that serve as the “vehicle” to provide the fat “dose”. Participants receive all of their food for each of the 2 four-week periods. Food is made or purchased and packed for participants by Diet Center Staff. Participants come to the Diet Center five times per week (Monday through Friday), eat one their meals of choice (under supervision), and take other meals/snacks that are packed for them to eat at a time and place of convenience. Meals for the weekend are packed out for consumption at home. Participants are instructed not to eat other foods. Dietary compliance checks will be done daily via questionnaire.

Primary endpoints are of the study are lipids and lipoprotein profile (TC, LDL-C, HDL-C, TG) (FIG. 4).

Data Analysis: Data is analyzed based on differences between the control, parent fat and the test fat. Standard methodology is employed to evaluate significant differences between the treatments for the endpoints and correlations between the various endpoints.

### Example 3

A Palm DAG and Palm Kernel DAG of the present invention were subject to compositional analysis.

General analytical methods for these analyses are as described in the American Oil Chemists' Society (AOCS) Methods, 4^{th} Edition (1990).

Appearance was assessed.

Moisture was assessed by a Karl-fisher test. A Karl-fisher test is a standard titration that quantifies trace amounts of moisture in a sample.

Free fatty acids were determined by the Ca 5a-40 method, as defined by the AOCS The peroxide value was determined by the Cd 8-53 method, also defined by the AOCS.

Positional analysis of fatty acid compositions was determined by pancreatic hydrolysis with sn-1,3 specific lipases.

To analyze sn-2 monoacylglycerides (MAGs), sn-1,3 positional fatty acids were detached from the glycerol backbone by enzymatic reaction sn-1,3 specific lipases. A reaction mixture containing sn-2 MAGs and the free fatty acids (FFAs) from the sn-1,3 lipase reaction was separated by thin-layer chromatography (TLC). sn-2 MAGs were collected from the TLC plate and analyzed by gas chromatography (GC) according to AOCS protocols for fatty acid composition.

Glyceride composition was also analyzed. To separate TAGs, 1,3-diacylglycerides, 1,2-diacylglycerides, MAGs and FFAs, high pressure liquid chromatography (HPLC) was carried out with an evaporative light scattering detector (ELSD). The results from this analysis were recalculated to present each glyceride as a percentage of the complete composition, based on a standard curve.

An SFA content in sn-2 MAGs of 28.8% was obtained as previously described.

The analysis was performed on an Agilent 1 100 HPLC system. The column was an Alltima Silica 5 u (250 mm×4.6 mm, 5 μL, by Alltech). The detector was an Alltech ELSD, and the analytical software was Chemstations.

TABLE 1
Fatty acid composition of Palm DAG (“Heartlite”)

| Appearance | a pale yellow solid | |
|---|---|---|
| Moisture & Impurities | 0.05% | |
| Free Fatty Acid | 0.15 | mgKOH/g |
| Peroxide Value | 0.2 | meg/kg |

| | |
|---|---|
| Typical Fatty Acids Composition* | |
| C14:0 | 1.3% |
| C16:0 | 46.0% |
| C16:1 | 0.5% |
| C18:0 | 4.3% |
| C18:1 | 35.8% |
| C18:2 | 8.8% |
| C18:3 | 0.1% |
| Total USFA (unsaturated fatty acid) | 54.2% |
| Total SFA (saturated fatty acid) | 51.5% |
| Glycerides contents | |
| Tri-acylglyceride (TAG) | 10.8% |
| Di-acylglyceride (DAG) | 88.9% |
| 1.3-diacylglyceride | 65.2% |
| 1,2-diacylglyceride | 23.7% |
| Mono-acylglyceride (MAG) | 0.2% |

*fatty acids are expressed in area %

TABLE 2
Fatty acid composition of Palm Kernel DAG

| Appearance | a pale yellow solid |
|---|---|
| Moisture & Impurities | 0.05% |
| Free Fatty Acid | 0.14 mgKOH/g |
| Peroxide Value | 0.05 meg/kg |
| Typical Fatty Acids Composition* | Fatty acids are expressed in area % |
| C8:0 | 1.1% |
| C10:0 | 1.9% |
| C12:0 | 47.0% |
| C14:0 | 18.3% |
| C16:0 | 9.3% |
| C16:1 | 0.1% |
| C18:0 | 2.5% |
| C18:l | 16.2% |
| C18:2 | 2.2% |
| Glycerides contents | AOCS official method CD 11d-96 |
| Tri-acylglyceride (TAG) | 19.8% |
| Di-acylglyceride (DAG) | 80.0% |
| 1.3-diacylglyceride | 57.6% |
| 1,2-diacylglyceride | 22.4% |
| Mono-acylglyceride (MAG) | 0.1% |

### Example 4

The solid fat index of the Palm Kernel DAG, unmodified palm kernel oil and unmodified palm oil were determined across a range of temperatures using a method based on AOCS Cd 10-57 (with modifications). The method can be used with oils and fats with a solid fat index of 50 or less at 10° C. The method can be used with margarine oils, shortenings, hydrogenated base stocks and other fats.

The method used to determine solid fat index empirically determines the melting profile of a fat under the conditions of the test. Solid fat index is calculated from the specific volumes associated with combined liquid and solid phases at specified temperatures, utilizing a calculated fat expansion/dilation in ml/kg of sample.

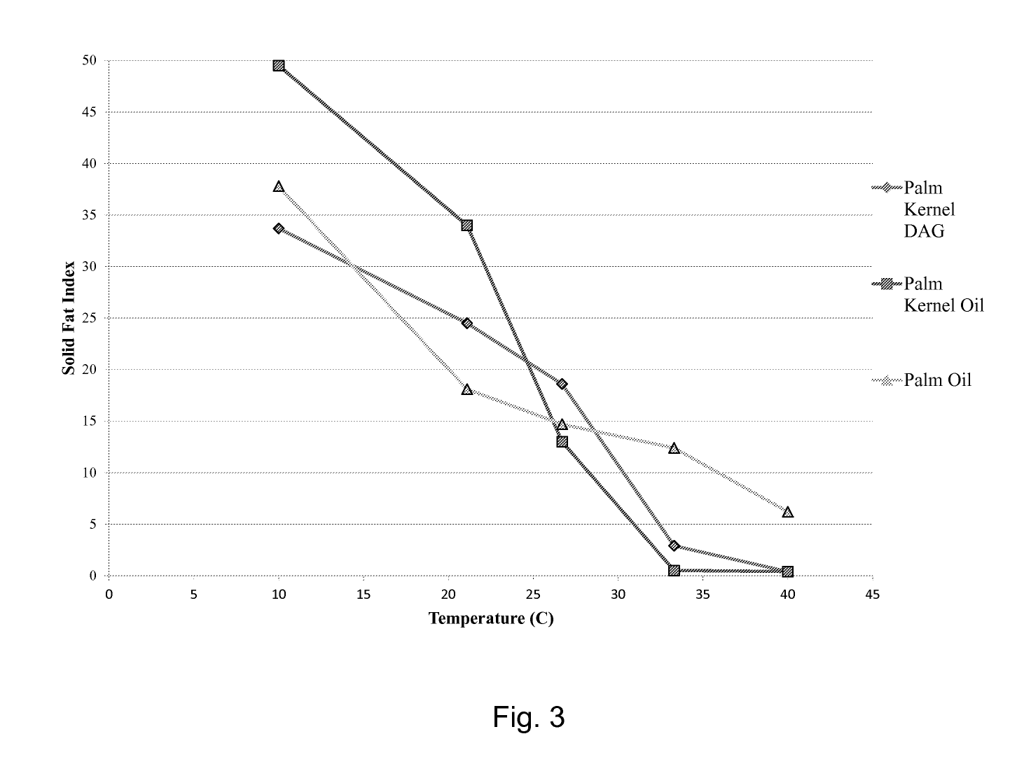

FIG. 3 shows in graphical form the data presented below in Tables 3-5. The y-axis shows the solid fat index of each of the three compositions. Temperature is plotted on the x-axis.

TABLE 3
Solid Fat Index of Palm Kernel DAG at various temperatures

| | |
|---|---|
| Temperature | Solid Fat Index |

| | | |
|---|---|---|
| 10° | C. | 33.7 |
| 21.1° | C. | 24.5 |
| 26.7° | C. | 18.6 |
| 33.3° | C. | 2.9 |
| 40° | C. | 0.4 |

TABLE 4
Solid Fat Index of Palm Kernel Oil at various temperatures

| | |
|---|---|
| Temperature | Solid Fat Index |

| | | |
|---|---|---|
| 10° | C. | 49.5 |
| 21.1° | C. | 34.0 |
| 26.7° | C. | 13.0 |
| 33.3° | C. | 0.5 |
| 40° | C. | 0.4 |

TABLE 5
Solid Fat Index of Palm Oil at various temperatures

| | |
|---|---|
| Temperature | Solid Fat Index |

| | | |
|---|---|---|
| 10° | C. | 37.8 |
| 21.1° | C. | 18.1 |
| 26.7° | C. | 14.7 |
| 33.3° | C. | 12.4 |
| 40° | C. | 6.2 |

The data above in Tables 3-5 and in FIG. 3 show that the Palm Kernel DAG composition disclosed herein has a favorable solid fat index compared to the control fats.

The solid fat index of the Palm Kernel DAG makes the DAG composition more useful for incorporation into foodstuffs than alternative fats currently in use. The Palm Kernel DAG composition has a preferable solid fat index when compared to alternative fats. This solid fat index profile allows use of less of the DAG composition to achieve the same texture as alternative fats.

The Palm Kernel DAG compositions of the present disclosure have a flatter solid fat index profile than other fats presently used in cooking. This flatter solid fat index profile allows the use of the compositions of the present disclosure in a wider range of temperatures than other fats.

The high melting point of the DAG composition can be useful in the creation or storage of foodstuffs that contain fat. Traditional compositions of many fat-containing foodstuffs can melt or become off-textured when prepared or stored at higher temperatures. Such “higher” temperatures may be only slightly “higher” than standard room temperature of approximately 25° C. Foodstuffs prepared with the DAG compositions of the present disclosure have an improved ability to be prepared at these “higher” temperatures as well as an enhanced shelf-life at such temperatures.

The Palm Kernel DAG compositions of the present disclosure have a higher solid fat index at lower temperatures and a lower solid fat index at higher temperatures. This combination of attributes allows use of the Palm Kernel DAG in shelf-stable foodstuffs, and simultaneously imparts a favorable melt-in-the-mouth texture when consumed. The low melting point (exemplified by a solid fat index of only slightly greater than O at only 40° C., Table 3) also allows more facile incorporation of the Palm Kernel DAG composition into foods, as it is easily fully melted.

### Example 5

Additional compositions of DAG-containing fats and oils are provided.

In one embodiment, DAG derived from palm oil is provided. The DAG can be 1,3-DAG or 1,2-DAG. The DAG can comprise from 15% to 99% SFAs. The DAG can comprise fatty acids selected from the group consisting of MUFAs, PUFAs, medium-chain fatty acids and a combination thereof.

In another embodiment, DAG derived from palm kernel oil is provided. The DAG can be 1,3-DAG or 1,2-DAG. The DAG can comprise from 15% to 99% SFAs. The DAG can comprise fatty acids selected from the group consisting of MUFAs, PUFAs, medium-chain fatty acids and a combination thereof.

In another embodiment, DAG derived from an oil from a tropical plant is provided. The DAG can be 1,3-DAG or 1,2-DAG. The DAG can comprise from 15% to 99% SFAs. The DAG can comprise fatty acids selected from the group consisting of MUFAs, PUFAs, medium-chain fatty acids and a combination thereof.

In a further embodiment, DAG derived from an oil derived from a temperate plant is provided. The DAG can be 1,3-DAG or 1,2-DAG. The DAG can comprise from 15% to 99% SFAs. The DAG can comprise fatty acids selected from the group consisting of MUFAs, PUFAs, medium-chain fatty acids and a combination thereof.

In an additional embodiment, DAG derived from an oil derived from an alga is provided. The DAG can be 1,3-DAG or 1,2-DAG. The DAG can comprise from 15% to 99% SFAs. The DAG can comprise fatty acids selected from the group consisting of MUFAs, PUFAs, medium-chain fatty acids and a combination thereof.

Compositions of the present invention include 1,2-DAG and 1,3-DAG where at the 1(3) and 2 positions, or at both the 1,2 and 1,3 positions can be SFAs of chain lengths between 8-18 carbon atoms. These SFAs can be derived from any source, for example but not limited to palm, coconut, any tropical oils, soy, sunflower and canola oils. Any of these oils can be modified to contain high SFA levels. In addition, the DAG compositions disclosed herein can comprise unsaturated fatty acids such as 18:1, 18:2, 18:3 (both omega 3 and omega 6), 18:4, 20:3, 20:4, 20:5 and 22:6 omega 3 fatty acids in the 1(3) or 2 positions of the DAG. These unsaturated FAs can be derived from any available source including fish, algal, and vegetable oils.

DAG-containing compositions of the present invention can be blended with other oils and/or fats to achieve desirable final compositions. Non-limiting examples of other oils and fats which could be blended with DAG-containing compositions include MUFAs, PUFAs, medium-chain fatty acids and a combination thereof. Oils and fats that can be blended with the DAG-containing compositions can be derived from any available source including fish, algae, and vegetables. Specific non-limiting examples of sources of oils include palm, coconut, any tropical oils, sunflower, corn, soybean, rapeseed and canola oils.

Specific non-limiting examples of fatty acids that can be included in DAG-containing blends include gamma-linolenic acid (γ-linolenic acid, “GLA”) and stearidonic acid. These fatty acids may themselves provide health benefits.

GLA is an 18:3 (omega-6) essential fatty acid. It is primarily found in plant-derived oils GLA may be able to suppress tumor growth and metastasis The lithium salt of GLA, Li-GLA, is in phase I1 clinical trials to determine whether it is useful in the treatment of HIV infections, since it has the ability to destroy HIV-infected T cells in vitro.

Eicosapentaenoic acid (EPA) supplementation has been shown to raise the omega-3 index and to lower risk for cardiac events. Stearidonic acid (also called moroctic acid) is an 18:4 (omega-3) essential fatty acid, and has been suggested as a source of omega-3 fatty acid that can raise EPA and/or docosahexaenoic acid (DHA) levels. It is biosynthesized from alpha-linolenic acid by the enzyme delta-6-desaturase. Sources of stearidonic acid include the seed oils of hemp, blackcurrant and echium, and the cyanobacterium spirulma.

The detailed description set forth above is provided to aid those skilled in the art in practicing the present invention. However, the invention described and claimed herein is not to be limited in scope by the specific embodiments herein disclosed because these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

### Example 6

A Palm DAG composition as described herein was subjected to compositional analysis.

General analytical methods used for this analysis were as described in Example 3.

TABLE 6
Fatty acid composition of Palm DAG

| Appearance | a pale yellow solid |
|---|---|
| Moisture & Impurities | 0.05% |
| Free Fatty Acid | 0.7 mg KOH/g |
| Peroxide Value | 0.95 meg/kg |
| Typical Fatty Acids Composition* | |
| C12:0 | 0.5% |
| C14:0 | 1.1% |
| C16:0 | 42.5% |
| C16:l | 0.2% |
| C18:0 | 9.5% |
| C18:l | 35.7% |
| C18:2 | 9.2% |
| C18:3 | 0.3% |
| Total USFA (unsaturated fatty acid) | 45.4% |
| Total SFA (saturated fatty acid) | 54.0% |
| Glycerides contents | |
| Tri-acylglyceride (TAG) | 4.8% |
| Di-acylglyceride (DAG) | 94.8% |
| 1.3-diacylglyceride | 79.6% |
| 1,2-diacylglyceride | 15.2% |
| Mono-acylglyceride (MAG) | 0.1% |

*fatty acids are expressed in area %

### Example 7

This study determined the effect of palm diacylglyceride oil (palm DAG oil; containing 89% DAG, with approximately 65% in the sn-1,3 form), compared with conventional palm oil (palm (TAG) oil), on low-density lipoprotein cholesterol in subjects with hypercholesterolemia. The Palm DAG used in this study was that described in Example 6. Food products were prepared as described in Example 1.

### Study Design

This pilot study was designed as a randomized, two-period blinded cross-over controlled feeding trial. Subjects received all food and drinks specific to the requirements for the study, and consumed sufficient calories to maintain their current body weight. During each 4-week treatment period, subjects consumed a standardized reduced-fat diet that included a specific amount of either palm DAG oil or palm oil. The diet containing palm DAG oil is referred to as the DAG diet, and the diet containing palm oil as the control diet. At the beginning of the study and at the end of each diet period, on two consecutive days, fasting blood samples (10-12 hr overnight fast), weight and blood pressure data were collected. A one week break was scheduled between each treatment period. If this break extended beyond two weeks an additional blood draw was performed before the start of the second diet period to re-establish a baseline. Compliance was assessed by daily monitoring of body weight, and completion of daily and weekly dietary intake records.

### Subjects

Healthy, non-smoking men and women (n=23) aged 30-60 years with moderately elevated LDL-C (120-175 mg/dL), and normal TG (<350 mg/dL) and HDL-C (>30 mg/dL) were recruited.

### Diets

Caloric requirements were initially determined by the Harris-Benedict equation, and adjusted accordingly to maintain body weight throughout the trial. Subjects consumed the same heart-healthy reduced-fat (American Heart Association (AHA) Step I; total fat <30% of energy, saturated fatty acids (SFA)<10% energy and cholesterol <300 mg/day) diet across the two, 4-week trial periods. These diets differed only by the addition of either palm DAG oil (herein called DAG diet) or palm oil (herein called Control diet), which was incorporated into recipes (i.e., yogurt, peanut butter, butter and cream cheese; see sample menus, Table 7). The palm DAG or palm oil consumed by subjects contributed ˜6.5% of total daily energy intake, with a minimum consumption of 15 g/day of palm DAG or palm oil. Subjects were instructed to consume only foods and beverages provided by the Diet Center, except for non-energy-containing beverages and seasonings, which were consumed ad libitum. Menus were created for a six day diet cycle across a range of calorie levels (1800-3000 kcal/day). The macronutrient compositions of the experimental diets (including the contributions from palm DAG oil or palm oil) are shown in Table 8.

TABLE 7
Sample daily menus for 2100 kcal experimental diets

BREAKFAST
Oatmeal
Blueberries, frozen
Healthy FAT- Peanut Butter
White sandwich bread
Apple Juice
Skim milk
LUNCH
Turkey breast, sliced
Mustard
White sandwich bread
Reduced fat provolone cheese
Healthy FAT- Yogurt
Pear
DINNER
Healthy Choice, Chicken Parmigiana
Dinner roll
Broccoli, frozen
Margarine
SNACK
Healthy FAT - Cream Cheese
Saltines
Apple Juice

TABLE 8
Macronutrient composition of the experimental diets¹

| MACRONUTRIENT | DAG Diet | Control Diet |
|---|---|---|
| Carbohydrate (% of energy) | 56.4 | 57.0 |
| Protein (% of energy) | 16.5 | 18.0 |
| Total fat (% of energy) | 28.2 | 28.5 |
| Saturated fat | 8.5 | 8.3 |
| Cholesterol (mg/d) | <100 mg/day | <100 mg/day |
| Dietary Fiber (g) | 26.8 | 27.2 |

¹Based on analyses of all foods used in a 6-day menu cycle for the 2100-kcal diets.

### Clinical and Laboratory Assessments

Body weight was measured at each laboratory visit (in addition to daily weigh-ins at the Diet Center). Blood pressure (3 repeats spaced 1 minute apart) was measured with subjects in a seated position after a minimum 5-min rest period, at each time point.

All blood samples were collected after an overnight (10-12 hr) fast according to a standardized protocol. Serum and plasma aliquots were stored at −80° C. Serum concentrations of lipids and lipoproteins (including second-day repeats) were measured using the VAP® (Vertical Auto Profile) test by Atherotech, Inc.

The VAP® Test provided a direct measure of the following lipid and lipoprotein classes and subclasses: LDL, LDL-real (LDL=LDL-real+Lp(a)+IDL), Lp(a), IDL (Intermediate-density lipoprotein), HDL, HDL2, HDL3, VLDL, VLDL1+2, VLDL3, TC, TG, Non HDL (=LDL+VLDL cholesterol), Remnant Lipoproteins, LDL4, LDL3, LDL2, ApoB100 (measure of total atherogenic particles in circulation), ApoA1 (measure of total anti-atherogenecity), ApoB100:A1.

All data were analyzed using SAS (STATISTICAL ANALYSIS SYSTEM, version 9.1.3; SAS Institute Inc, Cary, N.C.). Data were assessed for day to day variability, and hyper-variable cut-offs were established for LDL-C, HDL-C, TC and TG (12.5%, 10.4%, 9.1%, 25.6%, respectively). Day-to-day values that exceeded these percentages were identified and manually reviewed. Depending on their consistency and relationship with other values from the same individual, hyper-variable values were eliminated from the data set. A total of seven values were eliminated from the data set due to hyper-variability. Data were checked for distribution and log transformed as needed to achieve normality. Mixed models analysis (PROC MIXED) were used for exploratory analysis (α=0.05), with sex and diet (baseline, control and DAG) entered as factors for the analyses of lipids, lipoproteins, and other endpoint measures. This model used baseline lipid and lipoprotein values as a “diet” and not as a covariate. Change scores (Δ) were calculated by subtracting values at baseline from values at the end of each diet period, and analyzed by PROC MIXED.

### Results

Twenty-three subjects were enrolled in the study. Twenty subjects completed the study; 6 males and 14 females. Subject characteristics at baseline are shown in Table 9, including age, body mass index (BMI), TC, LDL-C, HDL-C, TG, systolic blood pressure (SBP), and diastolic blood pressure (DBP).

TABLE 9
Characteristics of study participants at baseline (mean ± SD).

| | |
|---|---|
| Age (y) | 48.85 ± 7.99 |
| BMI (kg/m²) | 27.55 ± 4.35 |
| Male:Female | 6:14 |
| TC (mg/dL) | 216.15 ± 24.99 |
| LDL-C (mg/dL) | 140.25 ± 20.78 |
| HDL- C (mg/dL) | 49.23 ± 14.14 |
| TG (mg/dL) | 136.95 ± 64.64 |
| SBP/DBP (mmHg) | 117.75 ± 11.3/80.65 ± 6.9 |

### Diet Effects

Table 10 shows the means and SEMs for each of the end-point measures at the beginning (baseline) and end of the diet intervention periods for all twenty subjects.

TABLE 10
Endpoint measurements at baseline and after the
experimental diets in all subjects (mean ± SEM).

| | P for | | | |
|---|---|---|---|---|
| | interaction | Baseline | Control Diet | DAG Diet |
| Weight (lb) | 0.92 | 173.1 ± 5.2 | 170.5 ± 5.1 | 170.6 ± 5.0 |
| SBP (mmHg) | 0.070 | 118.3 ± 2.7^{a} | 115.5 ± 3.3^{a,b} | 114.7 ± 2.8^{b} |
| DBP (mmHg) | 0.061 | 80.6 ± 1.6^{a} | 79.1 ± 2.3^{a,b} | 78.6 ± 1.7^{b} |
| TC (mg/dL) | 0.004 | 209 ± 5.2^{a} | 197.3 ± 5.3^{a,b} | 189.1 ± 4.8^{b} |
| LDL-C (mg/dL) | 0.0018 | 135.21 ± 4.3^{a} | 128.24 ± 4.7^{a,b} | 119.56 ± 4.2^{b} |
| LDL 1 | 0.0154 | 19.5 ± 1.2^{a} | 17.6 ± 1.1^{a,b} | 16.2 ± 1.2^{b} |
| LDL 2 | 0.0312 | 21.2 ± 2.6^{a} | 15.7 ± 2.4^{b} | 15.8 ± 2.4^{a,b} |
| LDL 3 | 0.1091 | 52.3 ± 3.4 | 50.9 ± 3.3 | 45.9 ± 3.1 |
| LDL 4 | 0.3058 | 18.4 ± 2.5 | 20.4 ± 2.6 | 18.4 ± 2.4 |
| HDL-C (mg/dL) | 0.0146 | 46.8 ± 2.5^{a} | 43.6 ± 2.4^{a,b} | 41.4 ± 2.4^{b} |
| HDL 2 | 0.0242 | 11.4 ± 1.1^{a} | 10.2 ± 1.0^{a,b} | 9.5 ± 0.9^{b} |
| HDL 3 | 0.0122 | 35.4 ± 1.4^{a} | 33.3 ± 1.5^{a,b} | 31.9 ± 1.4^{b} |
| VLDL (mg/dL) | 0.4154 | 26.8 ± 1.6 | 25.7 ± 1.3 | 27.5 ± 1.8 |
| VLDL 1 plus 2 | 0.2687 | 11.4 ± 0.9 | 10.8 ± 0.8 | 12.2 ± 1.2 |
| VLVL 3 | 0.5926 | 15.4 ± 0.8 | 14.8 ± 0.6 | 15.2 ± 0.7 |
| IDL (mg/dL) | 0.6704 | 17.1 ± 1.1 | 16.2 ± 0.9 | 16 ± 0.9 |
| Non-HDL (mg/dL) | 0.0371 | 162 ± 5.3^{a} | 153.7 ± 5.3^{a,b} | 147.5 ± 4.7^{b} |
| TC:HDL | 0.8741 | 4.7 ± 0.2 | 4.8 ± 0.2 | 4.8 ± 0.2 |
| LDL:HDL | 0.7664 | 3.1 ± 0.2 | 3.1 ± 0.2 | 3.0 ± 0.3 |
| TG (mg/dL) | 0.5956 | 128.2 ± 1.1 | 130.7 ± 1.1 | 138.5 ± 1.1 |
| Remnant Lipoproteins | 0.6950 | 32.6 ± 1.7 | 30.9 ± 1.3 | 31.1 ± 1.3 |
| LDL-real (mg/dL) | 0.0047 | 111.6 ± 4.2^{a} | 105.9 ± 4.6^{a,b} | 96.4 ± 4.6^{b} |
| ApoB100 | 0.0781 | 108.5 ± 3.3^{a} | 105.2 ± 3.2^{a,b} | 101.4 ± 3.3^{b} |
| ApoA1 | 0.0188 | 142.9 ± 3.9^{a} | 136.4 ± 4^{a,b} | 133.2 ± 3.8^{b} |
| ApoB100:ApoA1 | 0.7582 | 0.78 ± 0.03 | 0.79 ± 0.03 | 0.77 ± 0.03 |

Values in the same row with different superscripts are significantly different, adjusted p < 0.05.

### Lipids & Lipoproteins

Significant diet interactions were observed for TC, LDL-C (LDL 1 & 2), HDL-C (HDL 2 & 3) and Non-HDL cholesterol (Table 10). Post-hoc analyses showed that these variables were significantly reduced from baseline following the DAG diet, but not the control diet. However, the control and DAG diets were not significantly different.

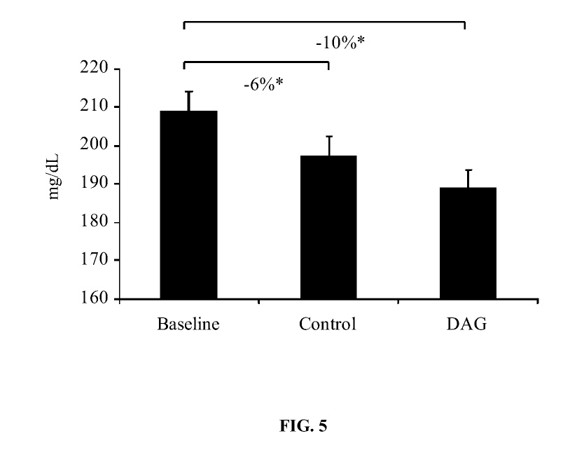

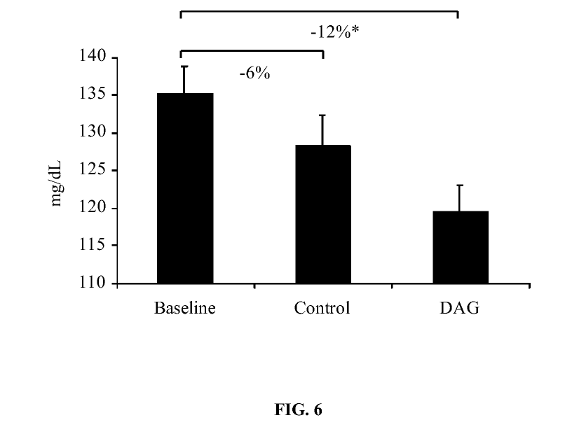

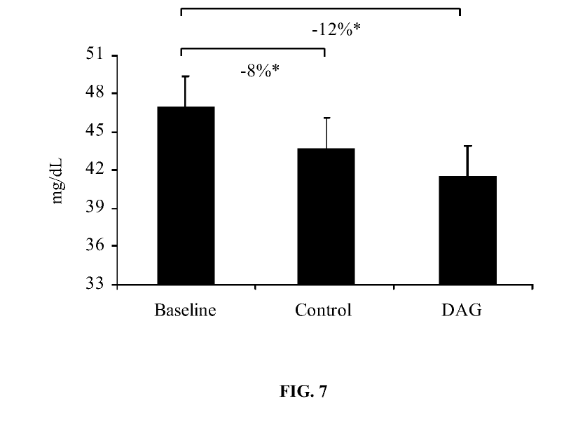

Percent change scores were calculated for TC, LDL-C and HDL-C to determine the magnitude of the reductions. Both TC and LDL-C were reduced by 6% following the control diet, whereas the DAG diet elicited a 10% and 12% reduction in TC and LDL-C, respectively (FIGS. 5 and 6). These changes in TC and LDL-C were significant for the DAG diet (ANOVA (analysis of variance); TC and LDL-C, p<0.01), but only TC on the control diet (ANOVA; TC p=0.016 and LDL-C, p=0.062). ANOVA of the change scores for total HDL-C showed that the DAG diet elicited a 12% (p<0.01) reduction from baseline, and the control diet an 8% reduction (p<0.01) (FIG. 7). Additional analyses showed that the change in HDL-C observed following the DAG diet was due to a significant shift in HDL 2 and 3, which were reduced by 17% and 10%, respectively (p<0.01). HDL2 and 3 also were significantly reduced by the control diet (p<0.01; HDL2, 12%; HDL3, 7%). The magnitude of the reduction between the two diets did not differ.

The effects of the intervention diets on ApoA1 mirrored those for HDL-C: ApoA1 levels were significantly lower following the DAG diet, but not the control diet, compared to baseline. There was a similar trend for a reduction in ApoB100 following the DAG diet, but not to the control diet (ANOVA, p=0.078).

### Blood Pressure

There was a strong trend toward an overall effect of diet on systolic and diastolic BP (SBP, p=0.07; DBP, p=0.06). The DAG diet lowered SBP and DBP by, on average, 3.6 mmHg and 2 mmHg, respectively from baseline. SBP was lowered by, on average, 2.8 mmHg and DBP by 1.6 mmHg following the control diet.

### Results

This study demonstrated that palm DAG oil incorporated within a heart healthy diet has beneficial effects on TC and LDL-C in healthy, hypercholesterolemic individuals. The outcomes of this study are in contrast to numerous other studies reporting that DAG oil reduces TG, but not TC or LDL-C in humans. For example, in Yamamoto, K. et al., J. Nutr. (2001) 131:3204-3207, substituting TAG cooking oil with DAG oil in Type II diabetic, hypertriglyceridemic men and women significantly reduced serum TG levels (35%), but total cholesterol and HDL-C did not change. Also, in Yamamoto K, et al., Nutrition (2006) 22:23-29, consumption of DAG oil (10 g/day) for three months lowered TG and increased HDL-C, ApoA1 and LDL particle size in Type II diabetics.

Although a reduction in TG following consumption of DAG oil was not observed in this study the proportion of carbohydrate (57%) in the diet may have negated these effects by maintaining higher levels of TG. The composition of the diets may also have influenced the study outcomes: both diets were lower in total (<30% calories) and SFA (<10% calories) than a standard Western diet.

Changes in ApoA1 mirrored the reductions in HDL-C: as ApoA1 is essential for HDL-C formation, these results are consistent with the lower HDL-C levels following the DAG diet. ApoA1 is involved in reverse cholesterol transport and is therefore considered to be anti-atherogenic. HDL-C and ApoA1 levels were lower in both the DAG and control diets. Total HDL-C at the end of both diet periods was above 40 mg/dL, the level recommended by the National Institutes of Health Adult Treatment Panel (ATP) III. Interestingly, there was a shift toward a reduction in HDL 2 and 3. HDL 2 is the largest and most buoyant HDL, and most protective against heart disease. Ideally, dietary interventions should maintain or increase HDL-C and HDL 2. There was a trend for a reduction in ApoB100 following the DAG diet compared to baseline, but not for the control diet. ApoB100 is the sole protein in LDL-C and the major protein in VLDL and IDL, so this trend is consistent with the observed reductions in LDL-C. ApoB100 plays a key role in LDL binding to specific receptors and in delivering cholesterol to tissue cells. Dietary interventions that lower ApoB100 may reduce risk for atherogenesis.

### Example 7

This postprandial study evaluated whether palm DAG oil reduced the hypertriglyceridemic and LDL-C response to a standardized meal, compared to a test meal containing conventional palm oil.

### Study Design

Participants were involved in this study for approximately 2-3 weeks. Postprandial challenges were conducted on two, single-day visits of approximately seven hours, which were spaced at least seven days apart. Prior to the first visit, participants were asked to eat a low fat diet for the 24 hours preceding the visit, and to refrain from taking vitamins or minerals for one week. To assist participants with their dietary intake, trained study staff provided general guidelines which met the current dietary recommendations of the American Heart Association; a diet high in fruits and vegetables, whole grains, low-fat dairy, and lean meats. In addition, subjects were asked to avoid fried foods, fast foods and other high fat foods. During the 24 hr period, participants recorded all the foods and drinks that they consumed, how much of the food they ate (e.g., 3 oz., 8 fl. oz., 1 C), and the brands of food (where possible) that were used. Food record logs were provided to assist in completing this task. Participants were asked to keep their diet and exercise relatively constant throughout the test period (approximately two weeks) to avoid significant weight loss or gain. Study staff reviewed each participants 24 hour diet record on the first day of testing and asked that they mimic as closely as possible this dietary pattern (low fat foods) during the 24 hours prior to the next scheduled test day.

### Subjects

Ten individuals who had completed the study in Example 6 within the previous six months were recruited. The same eligibility criteria used in that study was employed, eliminating the need for additional screening.

### Postprandial (PP) Challenges

The PP studies were conducted according to standardized protocols. Subjects were randomized to receive one of two test meals for the first PP test:
1. Control (conventional palm oil) meal: Cinnamon butter spread containing 30 g conventional palm oil served with four slices low calorie wheat bread.
2. Palm DAG meal: Cinnamon butter spread containing 30 g palm DAG oil (see Example 6) served with four slices low calorie wheat bread.
   The composition of the palm DAG cinnamon butter spread and the control cinnamon butter spread is shown in Table 11.

TABLE 11
Composition of palm DAG cinnamon butter
and control cinnamon butter

| | | |
|---|---|---|
| Ingredients | Grams | % by weight |

Palm DAG Cinnamon Butter

| | | |
|---|---|---|
| Palm DAG | 30.0 | 80.00% |
| Fat-free sweetened | 6.0 | 16.00% |
| condensed milk | | |
| Cinnamon | 0.75 | 2.00% |
| Pure vanilla extract | 0.75 | 2.00% |
| Total | 37.5 | 100.00% |

Control Cinnamon Butter

| | | |
|---|---|---|
| Palm oil | 30.0 | 80.00% |
| Fat-free sweetened | 6.0 | 16.00% |
| condensed milk | | |
| Cinnamon | 0.75 | 2.00% |
| Pure vanilla extract | 0.75 | 2.00% |
| Total | 37.5 | 100.00% |

Before each day of testing, subjects fasted overnight (at least 12 hours with no food or drink except water). On the morning of the test, subjects were weighed and their vital signs (temperature and blood pressure) taken before insertion of a catheter into the subject's arm. A baseline blood sample was drawn, and subjects were then asked to consume one of the two test meals described above within a fifteen minute period. No other food or drinks (other than water, caffeine-free diet soda, and diet jello) were allowed for the remainder of the testing period (6 hours).

Participants were asked to lie down or remain seated throughout the testing period. Sedentary activities such as reading, computer work, watching television or talking on the phone were allowed. Blood samples were taken at 30 minutes, 1, 2, 4, and 6 hours after consuming the meal (see timeline below; Table 12). At the end of the 6 hour period the catheter was removed and participants were briefly evaluated for safety before leaving the study site. This procedure was repeated at the second visit, where the 2^{nd} test meal was consumed.

TABLE 12
Timeline for PP study

| TIME | 8:00 am | 8:30 am | 8:45 am | 9:30 am | 10:30 am | 11:30 am | 1:30 pm | 3:30 pm |
|---|---|---|---|---|---|---|---|---|
| ACTION | Subject arrives | Catheter | Subject | BD | BD | BD | BD | BD |
| | at GCRC after | inserted. | consumes | (30 min | (1 hr post | (2 hr post | (4 hr post | (6 hr post |
| | overnight fast. | BD | test meal | post meal) | meal) | meal) | meal) | meal) |
| | | (baseline) | | | | | | |

BD = blood draw

### Laboratory Assessments

All blood samples were collected and processed for analyses according to standardized protocols.

### Statistical Analysis

All data were analyzed using SAS (STATISTICAL ANALYSIS SYSTEM, version 9.1.3; SAS Institute Inc, Cary, N.C.). Data were analyzed by two models: 1) repeated measures ANOVA (analysis of variance), where model=treatment, visit, time, baseline values; and 2) incremental (positive) Area Under the Curve (AUC). Data were checked for distribution and log transformed as needed to achieve normality.

### Results

Ten of the Example 6 study participants (5 females; 5 males) completed this postprandial (PP) study. Table 13 presents the baseline data for the Example 6 study participants (n=20) and the subset who completed the present PP study. On two occasions (time 240 and 360 min for one individual each) blood samples were not collected due to: 1) unsuccessful attempts to draw blood and 2) hemolysis of the samples. Complete data was available for 8 individuals (3 females; 5 males).

TABLE 13
Characteristics of study participants at baseline for the Example
6 and the subset of participants in the current study

| | | Example 7 |
|---|---|---|
| | Example 6¹ | (this study)² |
| Age (y) | 48.85 ± 7.99 | 46.7 ± 9.26 |
| BMI (kg/m²) | 27.55 ± 4.35 | 27.15 ± 2.81 |
| Male:Female | 6:14 | 5:5 |
| TC (mg/dL) | 216.15 ± 24.99 | 208.7 ± 21.58 |
| LDL-C (mg/dL) | 140.25 ± 20.78 | 134.5 ± 23.95 |
| HDL-C (mg/dL) | 49.23 ± 14.14 | 46.7 ± 13.75 |
| TG (mg/dL) | 136.95 ± 64.64 | 137.5 ± 52.79 |

¹Mean ± SD
²Baseline values for PP study are those collected at visit 1, time 0.

### LDL-C, TC, HDL-C

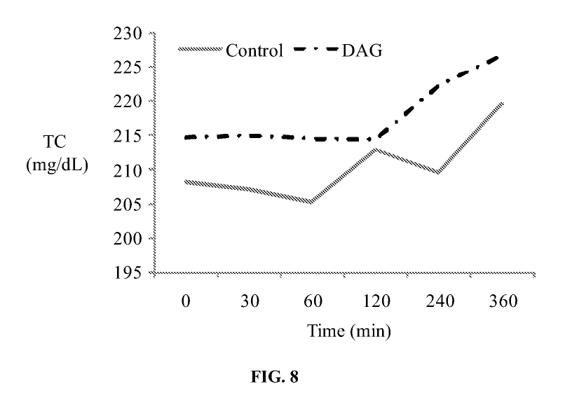

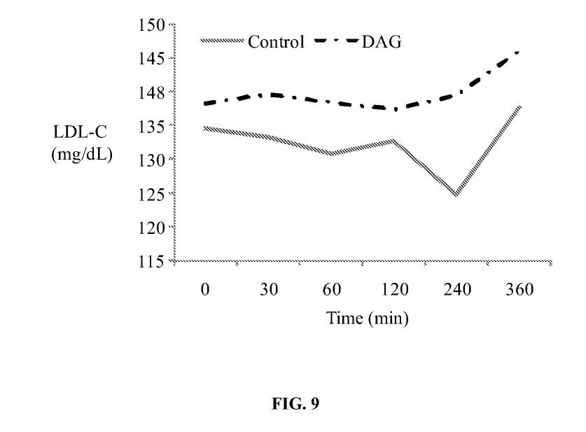

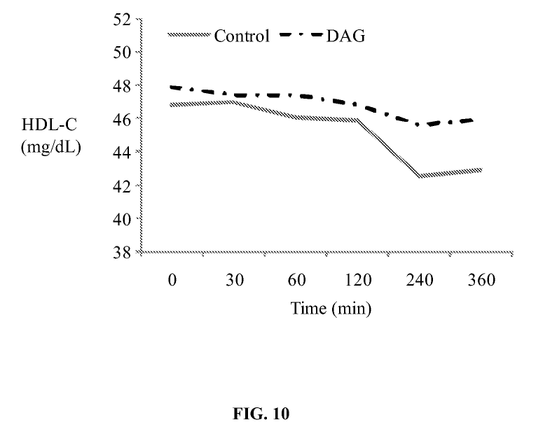

No treatment, visit or time effects were observed for TC, HDL-C, or LDL-C. Data are presented in FIGS. 8, 9, and 10.

### TG

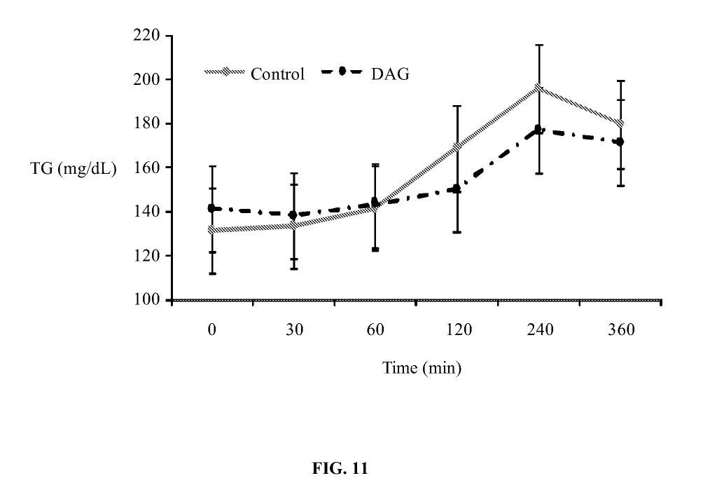

No significant treatment effects of DAG were observed for TG; but, the palm DAG oil elicited a small blunting effect on the PP TG response compared to the control meal (FIG. 11). Mean values are presented in Table 14.

TABLE 14
Mean (±SEM) TG (mg/dL) values after consumption
of palm DAG oil or control (palm oil)

| Time point (min) | Palm DAG | Control (palm oil) |
|---|---|---|
| 0 (baseline) | 141.4 ± 19.4 | 131.5 ± 19.4 |
| 30 | 138.5 ± 19.4 | 133.4 ± 19.4 |
| 60 | 142.8 ± 19.4 | 141.7 ± 19.4 |
| 120 | 150.4 ± 19.4 | 169 ± 19.4 |
| 240 | 177.2 ± 19.4 | 196.1 ± 20.1 |
| 360 | 171.6 ± 19.4 | 180 ± 20.1 |

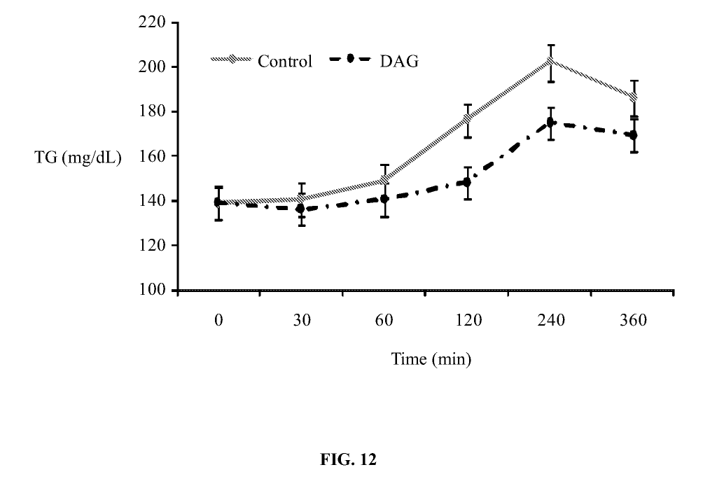

Because baseline TG values contribute to the variation in PP responses, the model was rerun adjusting for differences in baseline TG values between the control and DAG groups. The results are shown in FIG. 12. Mean adjusted values are presented in Table 15. In Model 2 there was a trend toward a significant reduction in the AUC variable for the TG response due to treatment. That is, the DAG treatment produced a lower AUC response compared to the control, which is consistent with the blunted response observed in Model 1.

TABLE 15
Mean (±SEM) TG (mg/dL) values after consumption
of palm DAG oil or control (palm oil).

| Time point (min) | Palm DAG | Control (palm oil) |
|---|---|---|
| 0 (baseline) | 138.7 ± 7.4 | 138.5 ± 7.4 |
| 30 | 135.8 ± 7.4 | 140.4 ± 7.4 |
| 60 | 140.1 ± 7.4 | 148.7 ± 7.4 |
| 120 | 147.7 ± 7.4 | 176.0 ± 7.4 |
| 240 | 174.5 ± 7.4 | 202.0 ± 8.2 |
| 360 | 168.9 ± 7.4 | 185.9 ± 8.2 |

Values are controlled for baseline TG levels.

### Discussion

In summary, the palm DAG oil blunted PP TG response but had a neutral effect on TC, LDL-C and HDL-C. Similar to other studies, the beneficial effect of DAG on PP TG levels is more pronounced in the later hours (>240 min), indicating a significant influence of time on PP response. DAG seems to have little or no effect on serum cholesterol (TC, LDL-C, HDL-C) concentrations in both fasting and postprandial human studies.

### Example 8

This postprandial study evaluated whether palm DAG oil reduced TC, LDL-C, HDL-C, or TG following administration of a meal containing a palm DAG composition, compared to a test meal having conventional palm oil.

### Study Design

This study was conducted as a blinded randomized cross-over trial. Subjects had no knowledge about the nature of the fats administered.

### Subjects

Twenty subjects participated in the study (10 women, 10 men). Their average baseline data is presented in Table 16.

TABLE 16

| Characteristics | Mean ± SD |
|---|---|
| Age (yr) | 28.55 ± 10.99 |
| Gender | |
| Male | 10 |
| Female | 10 |
| Total cholesterol (TC) (mmol/L) | 4.56 ± 0.67 |
| HDL-C (mmol/L) | 1.45 ± 0.38 |
| LDL-C (mmol · L) | 2.66 ± 0.45 |
| TG (mmol/L) | 0.84 ± 0.25 |
| Glucose (mmol/L) | 4.89 ± 0.40 |
| BMI (wt/ht²) | 24.79 ± 5.36 |

### Diet

Subjects reported to the study clinic after an overnight fast (minimum 10 hours) and a fasted blood sample was drawn from each volunteer (zero, 0 h). They were then provided the fat challenge in the form of breakfast including four slices of low-glycemic bread and 40 g of the Heartlite or control fat in the form of peanut butter. The meal was accompanied by mineral water only. Consumption of breakfast was completed within 15 minutes. Thereafter, blood was drawn at timed intervals of 30, 60 and 90 minutes, 2 h, 4 h, 6 h, 8 h. At the end of 8 h, the study ended and all volunteers were provided a cooked meal.

No other food was consumed throughout the postprandial duration of 8 h. Volunteers had free access to mineral water only. Through this procedure it was possible to ensure that the fat load for each subject was primarily from the test or control fats. It is estimated that the palm DAG or control fat provided nearly 85% of the subject's fat intake since fat from other food sources, namely the low-glycemic bread and condensed milk added to the peanut butter, did not significantly contribute to the fat load. This estimate was confirmed by food analysis (see Table 17). This approach allowed for the conclusion that the postprandial outcomes reported here are primarily fat mediated effects.

TABLE 17
Fatty Acid Compositions of Palm DAG and Conventional Palm Oil
compared to the Fatty Acid Composition of the test meal.

| | | | |
|---|---|---|---|
| 16:0 | 18:0 | 18:1 | 18:2 |

Fatty Acid Composition* (%) of Palm DAG and Conventional Palm Oil

| | | | | |
|---|---|---|---|---|
| Palm Oil | 42.77 | 5.33 | 40.29 | 10 |
| Palm DAG | 40.49 | 8.89 | 37.71 | 11.14 |

Fatty Acid Composition of Breakfast Consumed by Subjects

| | | | | |
|---|---|---|---|---|
| A1 - Palm | 42 | 5.43 | 38.91 | 11.67 |
| A2 - Palm | 42.51 | 5.25 | 38.7 | 11.83 |
| B1 - Palm DAG | 40.32 | 8.97 | 37.03 | 11.81 |
| B2 - Palm DAG | 40.23 | 8.83 | 37.08 | 11.89 |

*Only major fatty acids are reported here.

### Results

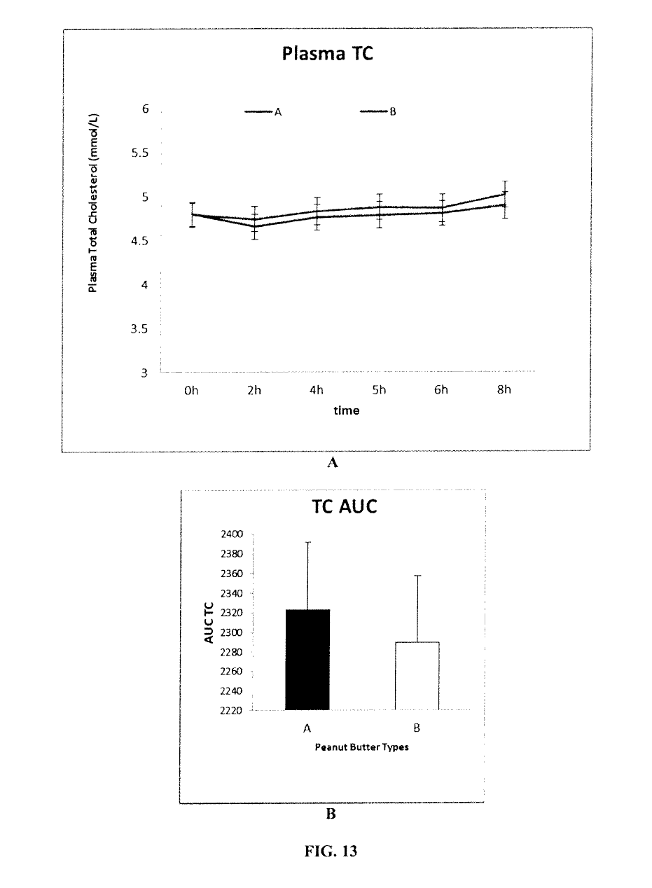

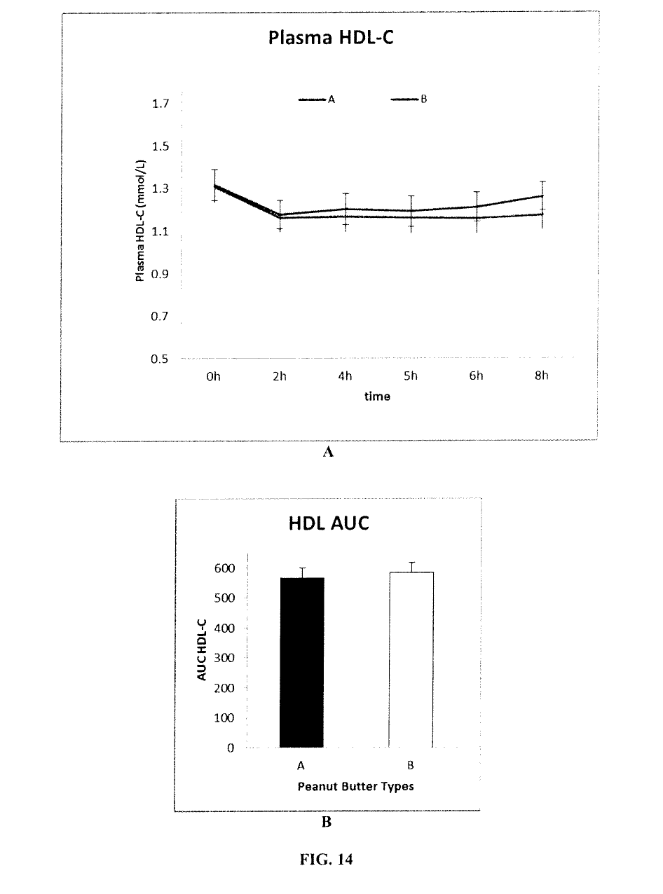

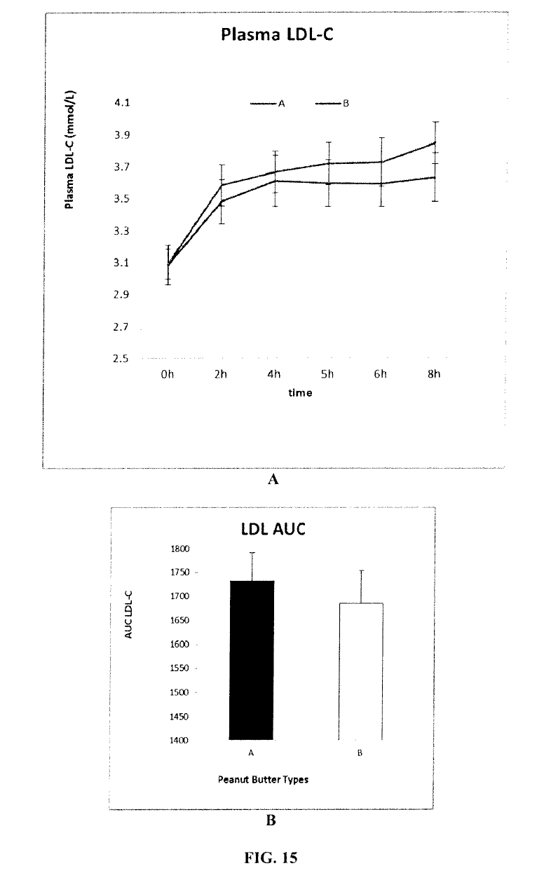

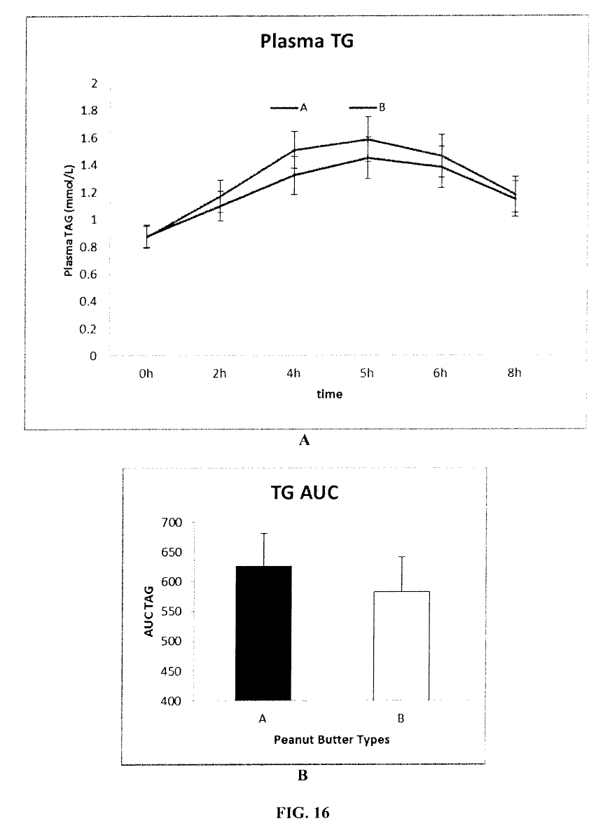

As is shown in FIGS. 13-16, postprandial lipid responses for TC, TG, LDL-C and HDL-C tended to favor the group administered a meal containing the palm DAG fat composition.

### REFERENCES CITED

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

What is claimed is:

1. A fat composition comprising from about 20% to about 100% by weight of diacylglycerols derived from a tropical oil.

2. The fat composition of claim 1, wherein said tropical oil is selected from the group consisting of palm oil, palm kernel oil, cocoa oil, shea oil, and coconut oil.

3. The fat composition of claim 2, wherein said tropical oil is selected from palm oil and palm kernel oil.

4. The fat composition of claim 3, wherein said palm oil is a fraction of palm oil selected from palm olein, palm stearine, fractionated palm olein, palm mid-fraction, and mixtures thereof.

5. The fat composition of claim 3, wherein said palm kernel oil is a fraction of palm kernel oil selected from palm kernel olein, palm kernel stearin, and mixtures thereof.

6. The fat composition of claim 1, wherein said composition exhibits beneficial health effects when an effective amount of said composition is ingested by a mammal for at least 4 weeks.

7. The fat composition of claim 6, wherein said beneficial health effect is selected from the group consisting of lowered serum LDL and lowered total serum cholesterol.

8. The fat composition of claim 1, wherein said composition has a solid fat index (SFI) of at least 15% at room temperature.

9. The fat composition of claim 1, wherein said composition comprises from about 40% to about 100% by weight of said diacylglycerols.

10. The fat composition of claim 9, wherein said composition comprises from about 70% to about 100% by weight of said diacylglycerols.

11. The fat composition of claim 10, wherein said composition comprises from about 85% to about 100% by weight of said diacylglycerols.

12. The fat composition of claim 1, wherein said diacylglycerols comprise at least 25% by weight 1,3-diacylglycerols.

13. The fat composition of claim 12, wherein said diacylglycerols comprise at least 50% by weight 1,3-diacylglycerols.

14. The fat composition of claim 12, wherein said diacylglycerols comprise from about 50 to about 80% by weight 1,3-diacylglycerols.

15. The fat composition of claim 1, wherein said composition comprises less than about 1% by weight of one or more of a phytosterol, phytostanol, phytosterol ester, or phytostanol ester.

16. The fat composition of claim 15, wherein said composition comprises no detectable level of one or more of a phytosterol, phytostanol, phytosterol ester, or phytostanol ester.

17. A food composition comprising a fat composition as set forth in claim 1, wherein said food composition is selected from the group consisting of: cakes, pies, breads, muffins, crackers, sweet dough, pastry, cream filling, yogurt, pudding, cream cheese, spreads, salad dressing, margarines, butter, mayonnaise, frozen foods, ice cream, frozen desserts, frozen yogurt, smoothies, peanut butter, granola, bars, cookies, and medical foods.

18. The food composition of claim 17, wherein said food composition exhibits one or more enhanced characteristics selected from the group consisting of enhanced shelf-stability, enhanced emulsion stability, reduced brittleness, enhanced spreadability, enhanced melt-in-the-mouth sensation, higher melting-point, reduced trans fatty acid content per unit of solids consumed, reduced susceptibility to oxidation, enhanced texture, enhanced palatability, enhanced lubricity, and enhanced air trapping capacity.

19. The food composition of claim 17, wherein said medical food is a meal replacement bar, meal replacement beverage, semi-solid beverage for enteric nutrition, or a semi-solid beverage for tube feeding.

20. An oil composition comprising from about 20% to about 100% by weight of diacylglycerols derived from a temperate oil, said temperate oil having a saturated fatty acid content of at least 15%.

21. The oil composition of claim 20, wherein said temperate oil is selected from the group consisting of corn oil, soybean oil, canola oil, and sunflower oil.

22. A fatty acid composition comprising from about 20 to about 95% by weight of diacylglycerols, wherein said diacylglycerols comprise 1,2-diacylglycerols and 1,3-diacylglycerols, and wherein from about 15% to about 40% by weight of said diacylglycerols comprise at the 2 position, an unsaturated fatty acid, and wherein from about 25% to about 100% by weight of said diacylglycerols comprise at the 1(3) position, a saturated fatty acid having a chain length of 6-18 carbon atoms.

23. The fat composition of claim 22, wherein said unsaturated fatty acid is from a source selected from the group consisting of fish oil, algae, a temperate oil, and combinations thereof; and wherein said saturated fatty acid is from a source selected from the group consisting of palm, palm kernel, coconut, sunflower, corn, soybean, canola, and high stearate oils.

24. The fat composition of claim 22, wherein said unsaturated fatty acid is selected from the group consisting of 18:1, 18:2, 18:3, 18:4, 20:3, 20:4, 20:5 and 22:6 fatty acids.

25. The fat composition of claim 22, wherein said unsaturated fatty acid comprises gamma-linolenic acid or stearidonic acid.

26. A fat composition consisting essentially of from about 60 to about 100% by weight of diacylglycerols, wherein the fatty acid content of said diacylglycerols consists essentially of from about 40 to about 50% by weight 16:0 fatty acid, from about 3 to about 6% by weight 18:0 fatty acid, from about 30 to about 45% by weight 18:1 fatty acid, and from about 7 to about 12% by weight 18:2 fatty acid.

27. The fat composition of claim 26, wherein said diacylglycerols are derived from palm oil.

28. A fat composition consisting essentially of from about 60 to about 100% by weight of diacylglycerols, wherein the fatty acid content of said diacylglycerols consists essentially of from about 45 to about 55% by weight 12:0 fatty acid; from about 15 to about 20% by weight 14:0 fatty acid; from about 6 to about 12% by weight 16:0 fatty acid; and from about 10 to about 20% by weight 18:1 fatty acid.

29. The fat composition of claim 27, wherein said composition consists essentially of from about 85 to about 90% by weight of said diacylglycerols.

30. The fat composition of claim 27, wherein said diacylglycerols comprise from about 60 to about 80% by weight 1,3-diacylglycerols and from about 20 to about 40% by weight 1,2-diacylglycerols.

31. The fat composition of claim 27, wherein said composition consists essentially of less than about 10% by weight monoacylglycerols.

32. The fat composition of claim 27, wherein said composition consists essentially of less than about 15% by weight triacylglycerols.

33. The fat composition of claim 27, wherein the fatty acid content of said diacylglycerols comprises about 46% by weight 16:0 fatty acid, about 4% by weight 18:0 fatty acid, about 36% by weight 18:1 fatty acid, and about 9% by weight 18:2 fatty acid.

34. The fat composition of claim 28, wherein said diacylglycerols are derived from palm kernel oil.

35. A method of lowering total serum cholesterol or serum LDL in a mammal, said method comprising administering to said mammal an amount of said composition of claim 1 or claim 20 to provide at least 15 g/day of diacylglycerols to said mammal and for a period of time sufficient to lower said total serum cholesterol or serum LDL.

36. The method of claim 35, wherein said palm oil is a fraction of palm oil selected from palm olein, palm stearine, fractionated palm olein, palm mid-fraction, and mixtures thereof.

37. The method of claim 36, wherein said palm kernel oil is a fraction of palm kernel oil selected from palm kernel olein, palm kernel stearin, and mixtures thereof.

38. The method of claim 35, wherein said composition is administered as an ingredient in a foodstuff.

39. The method of claim 35, wherein said composition is administered as an ingredient in a medical food selected from the group consisting of a meal replacement bar, meal replacement beverage, semi-solid beverage for enteric nutrition, and a semi-solid beverage for tube feeding.

40. The method of claim 38, wherein said foodstuff is selected from the group consisting of: cakes, pies, breads, muffins, crackers, sweet dough, pastry, cream filling, yogurt, pudding, cream cheese, spreads, salad dressing, margarines, butter, mayonnaise, frozen foods, ice cream, frozen desserts, frozen yogurt, smoothies, peanut butter, granola, bars, and cookies.

41. The method of claim 38, wherein said ingredient is selected from the group consisting of: a shortening, bakery fat, frying fat, coating fat, non-dairy fat, cocoa-butter equivalent, butter, margarine, and vanaspati ghee.

42. The method of claim 38, wherein said ingredient contains less than about 1% by weight phytosterol, phytostanol, phytosterol ester, or phytostanol ester.

43. The method of claim 35, wherein said mammal has been diagnosed as having a cardiovascular disorder, type II diabetes, or metabolic syndrome.

44. The method of claim 43, wherein said cardiovascular disorder is selected from one or more of: hypertriglyceridemia, hypercholesterolemia, other hyperlipidemias, hyperglycemia, hyperinsulinemia, arteriosclerosis, atherosclerosis, arteriolosclerosis, angina pectoris, thrombosis, myocardial infarction, and hypertension.

45. The method of claim 35, wherein said mammal has a serum level of LDL ranging from about 120 to about 175 mg/dL prior to administration of said composition.

46. The method of claim 35, wherein said lowering occurs after administering said composition for at least twelve weeks.

47. The method of claim 35, wherein said method comprises administering to said mammal an amount of said composition to provide at least 30 g/day of diacylglycerols.

48. The method of claim 35, wherein said method comprises administering to said mammal an amount of said composition to provide at least 40 g/day of said diacylglycerols.

49. A method for producing a food product, said method comprising substituting diacylglycerols derived from palm oil, a palm kernel oil, or a mixture thereof for about 5% to about 100% of a hydrogenated fat or oil in said food product.

50. A method of making a composition, said method comprising:
a) reacting free fatty acids and glycerol with one or more sn-1,3-regiospecific lipases, wherein said free fatty acids are from a tropical oil or a temperate oil having a saturated fatty acid content of at least 15%; and
b) purifying the resulting diacylglycerol products to obtain said composition having at least 20% diacylglycerols.

51. The method of claim 50, wherein said one or more sn-1,3-regiospecific lipases are immobilized.

52. The method of claim 50, wherein said tropical oil is selected from palm oil and palm kernel oil.

53. The method of claim 50, wherein said temperate oil is selected from corn, soybean, canola and sunflower oil.
